SEQUENCE LISTING (30 sequences)

SEQ ID NO: 1 - DNA, 10 bp, Artificial
  Arbitrary decamer primer
acaaggcacc

SEQ ID NO: 2 - DNA, 10 bp, Artificial
  Decamer anchor primer
ttttttttcg

SEQ ID NO: 3 - DNA, 24 bp, Artificial
  Drop1 Upper Primer
ctggagaaacggctgtcacaaata

SEQ ID NO: 4 - DNA, 28 bp, Artificial
  Drop1 Lower primer
cctctctacatagtcattccattggcta

SEQ ID NO: 5 - DNA, 33 bp, Artificial
  Primer U278
gtcgacgatggcaacctccagaggggcctcccg

SEQ ID NO: 6 - DNA, 31 bp, Artificial
  Primer L1492
gggccctggtgcccagaggtgcaggaagaga

SEQ ID NO: 7 - DNA, 33 bp, Artificial
  Primer
gatatcatgtatttagatgcggtccacgagttc

SEQ ID NO: 8 - DNA, 34 bp, Artificial
  Primer
gtcgactggggacccttgatttagaggttaactt

SEQ ID NO: 9 - DNA, 10000 bp, Homo sapiens
cggccgccggccgcagcgggctgagattgttgtcctctgtcaccagggcggctgggctcccgcagtcctgcagaccgcgcccgatcccggcgacagggcgggcggacagccgcgcatccccggggtcccgccgagcctgggcgcagagagccgggaggaagcgttcgctcgcttcgccttgctgctgggaaactgaacgaggccgagagagaaggttcttgagttcatgtaagaggacagtcttaaaacggaagaagaaaaagaagcagttcagtctttgggagagctgcctccttgttgagtgctgcaaaggcctggaattcatttatgacagaatagatctagaaaagtccaagcatgttttctagagtggtgtagccctgtgctgcctccagtgaagagtctcttggtgttggcttcgtgcttccggagggaccatggcaacctccagaggggcctcccggtgtcctcgggatatcgccaatgtgatgcagaggctgcaagatgagcaagagatagtacaaaaacgaactttcacaaaatggatcaactctcatctggccaagcggaaacctccaatggtggtggacgatctttttgaagacatgaaagatggtgttaaactgcttgcccttctggaggtcctgtctgggcagaaactgccttgtgaacaaggacgccggatgaagcgaatccatgctgtggctaacattggcacggcactcaagttcctcgaaggaagaaagtccatgcacagaggatcaccgattaaattagtcaacattaactccaccgatatagctgatggccgaccctcaatagttcttggattgatgtggaccattattctatatttccagattgaagagttgaccagcaacctgccccagctccagtctttgtccagcagcgcatcctccgtggacagcatagttagctctgagactcccagcccaccaagtaaacggaaggtgaccaccaagatccaaggaaatgctaagaaggctttattaaagtgggttcagtacacagctggcaagcagactggaatagaagtaaaagattttgggaagagttggagaagcggggttgcctttcattcagttattcatgccattcgaccggaattggtggacttggagacagtgaaaggcagatccaaccgagaaaatttggaggatgctttcactatcgccgaaacagaactggggatcccaagactgctagatcctgaagacgttgatgtggataaaccagatgagaaatctattatgacctatgtagcccagtttctgaaacattatcctgacatccacaatgcaagcactgatgggcaagaggatgatgaaatacttccaggtttcccatcttttgcaaattctgtacaaaattttaagagagaagacagagtaatttttaaggaaatgaaagtttggatagaacaatttgagagagatttgacaagagcacagatggtggaatcaaatttacaggataaatatcagtcatttaagcacttcagagttcaatatgaaatgaagaggaaacagattgaacatttaatacaaccattacacagagacggtaaattgtcacttgaccaagcattggtaaaacaatcttgggatagagtgacctccaggctctttgactggcatatacagcttgataaatctcttcctgcacctctgggcaccataggtgcctggctgtacagagcggaggtggccctgagagaggaaataaccgttcaacaggtccacgaggaaacagcaaacacgatacaacggaaacttgagcaacataaggatctgcttcaaaacacggatgcccacaaaagagcattccatgaaatctaccggaccaggtctgttaacgggattccagtgccacctgatcaattagaggacatggccgagaggtttcattttgtttcctccacatcagagctacacctaatgaaaatggaatttttagaattaaagtaccgtctgctctcactgctggttcttgcagagtcaaagctgaagtcttggatcattaagtacgggaggagagagtcagtggagcagcttctacaaaactacgtgtcttttatagaaaatagcaagttctttgaacaatatgaggtgacataccagatcttgaaacagacagctgagatgtatgtcaaagcagatggttcagtggaagaagctgagaatgtgatgaaattcatgaatgaaaccaccgctcagtggaggaatctctcagtagaagtgaggagtgtgaggagcatgctggaagaagtgatctctaactgggatcgctatggcaatacagtggctagtctgcaagcctggctagaggatgctgaaaaaatgctcaatcaatcagaaaatgccaaaaaggatttttttcgaaatttacctcattggattcagcagcatactgccatgaacgatgctggcaattttctaattgaaacctgtgatgagatggtttcccgtgacctgaagcagcaattactgttgctaaatgggcggtggagggagttgtttatggaagtcaagcaatatgctcaagctgatgagatggacagaatgaagaaggaatacacagactgtgttgttaccctgtctgcttttgcaacggaagcccataagaaactttctgaacccttagaagtctcttttatgaatgtcaagctattaattcaagacttggaggatattgagcagagggtgcctgtgatggatgcccaatacaagataattacaaagacagcacacctcattaccaaagaaagcccccaagaagaaggaaaagaaatgtttgcgaccatgtcaaagctcaaagagcagctaaccaaggtcaaagaatgttactccccactcctttatgagtctcagcagctgttgattccgttggaggaattagaaaagcagatgacgtccttttatgactcacttgggaaaatcaatgaaattatcacagttcttgagcgtgaggcacaatcgagtgccctttttaaacaaaaacatcaggaactgttagcttgtcaagaaaactgtaagaaaaccttgacacttattgagaaaggcagtcaaagtgttcaaaagtttgtgaccttgagcaacgtgttaaagcattttgatcagacgaggctacaaagacagattgcagatattcatgttgcttttcagagtatggtaaagaaaactggagattggaagaagcatgtggaaaccaacagtcgcttgatgaagaagtttgaggagtctcgagcagagttggagaaggtactgcggattgctcaggagggcctggaggaaaagggggatccagaggagctcctgcggagacacactgagtttttcagtcagctggatcagagggtgctcaatgctttcctgaaagcttgtgatgaactcaccgacatccttccagagcaggagcagcaggggctgcaggaagctgttcgaaagctccacaaacaatggaaggatcttcaaggagaagccccttatcatttgcttcatctgaagattgatgtggagaagaataggttcttagcctctgtagaagaatgcagaactgagctggatcgagagaccaagctgatgccccaggaaggcagtgaaaagataattaaagagcacagggttttcttcagtgacaaaggtcctcatcatctctgtgagaaaaggttacagctcatcgaggaactctgtgtgaaactcccagtgcgggacccagtaagggacacacctggaacctgtcacgtgactctcaaagagctcagagctgccattgacagcacctacaggaagctcatggaagacccagacaagtggaaggactacactagcagattctctgagttctcatcttggatatctacaaatgagacacaattaaaggggatcaagggtgaggccatcgatactgccaaccacggagaggttaaacgtgccgttgaagagatcagaaatggtgttaccaaaaggggtgagaccctcagctggctgaaatccaggctgaaagttttgacagaagtttcttctgagaatgaagcccaaaagcagggagatgagctggcaaaattatccagctctttcaaggctcttgtgacgctgctgtcagaggttgaaaagatgctaagcaattttggggactgtgtccagtacaaagaaatagtcaaaaattctctcgaagaattaatttctggctctaaagaagtccaggaacaagctgagaagatcttggatactgaaaatctgtttgaagcacagcagttacttcttcatcaccagcaaaagacaaagcggatctcagcaaagaagagagatgtgcagcagcagatcgcgcaggcgcagcagggagaaggggggctgcctgaccgaggccacgaggagctgcggaagctggagagcacactggatggcctggagcgcagccgggagaggcaggaacgccgcatccaggtcacattaagaaaatgggagcgatttgaaacaaacaaagaaacagtagtaagatacctttttcaaacaggttccagtcatgaacgcttcttgagttttagcagtttggaaagtttatcttcagaactggaacaaacaaaggagttttctaaacggacagaaagtattgcagtccaggctgagaaccttgtaaaggaagcttcagagataccgcttgggccccaaaataagcagctgcttcaacagcaggccaagtcaatcaaagaacaagtcaaaaaattagaagacacgcttgaagaagatattaaaaccatggaaatggtgaaaaccaagtgggatcattttggcagtaattttgagactctgtccgtctggataactgagaaagaaaaagaactcaatgccttggaaacttcgtcatctgccatggacatgcaaatcagccaaattaaggtcacaattcaggaaatagaaagtaagctcagcagcattgtaggattagaagaagaagcccagtcttttgctcagtttgttaccactggagaatctgctcgaattaaagccaagttgacacaaataagaagatacggggaagagcttcgagagcatgcacagtgtctggaaggaacaatcctgggacatttatctcagcagcaaaagtttgaagagaaccttagaaagatccagcaatctgtgtctgaatttgaagataaacttgctgttccaattaaaatatgttcttcagctacagaaacatacaaagttcttcaagaacatatggatctctgccaggccctggagtcactgagcagcgcgatcactgccttctcagccagtgccaggaaggttgtgaacagagattcctgtgttcaggaggctgcggctctacagcagcaatacgaggacatcctaaggagggcgaaggagagacagacggcgctggagaatctgctggcccactggcagaggctagagaaagaactatcatcctttttgacctggttagagcggggtgaagctaaagccagttccccagaaatggacatttctgcagacagagtcaaagtggaaggtgaacttcagttaatacaggcactgcaaaatgaagttgtatcccaggcctcattctatagcaaacttttgcaattgaaggaatcattgttctcagtagcctccaaagatgatgtgaaaatgatgaaactacatttggagcagttggatgagagatggagagatttaccacagatcattaacaaaaggattaattttcttcagtctgtggttgctgaacaccagcaatttgatgagctgctgctttccttttctgtctggattaaactgtttctcagtgaattacaaactacctctgagattagcataatggaccatcaagtagcccttactcggcataaggaccacgcagcagaagtagagagcaaaaagggcgaattgcagagtctgcagggtcacttagcaaagttgggttctctgggccgtgctgaggacctccacctcctgcagggaaaggctgaggactgcttccagctgtttgaggaggccagccaggttgtggagaggcggcagcttgccctgtcccatttggcagaattcctccagagccatgcctctctgtccggcattctccgccagctgaggcaaacagtggaagcaaccaacagtatgaataagaacgagtctgatttgatagaaaaggacctcaatgatgctcttcaaaatgctaaagcattagaatctgctgccgtcagtctggatggcattctttccaaagcccaataccatctgaaaatcgggagctctgagcaaaggacttcctgcagagccacggctgatcagctctgtggagaggtagagaggatccagaaccttctgggaaccaagcagagtgaggcagatgctctggcagtgttgaaaaaagcattccaagaccagaaagaggagcttctgaaaagcattgaggacattgaagaaaggactgacaaagagcgattgaaagaacctacccgccaagctcttcagcagaggttaagagtgtttaatcagctagaagatgaattgaattctcacgagcatgaactatgttggttgaaagacaaagccaagcaaattgcccagaaagatgtagcttttgcacctgaagttgacagggagataaaccgcttagaggtcacctgggatgataccaaaagactaattcatgaaaatcagggtcagtgctgtggacttattgacttaatgagagaatatcagaacctgaaatcagctgtatctaaagtcttagaaaatgccagcagtgtgattgtaaccagaactaccataaaagatcaggaggatcttaaatgggctttttccaagcatgaaactgccaagaacaaaatgaattacaaacagaaagacttggataactttaccagcaaaggaaaacacttgttatctgagctgaagaaaattcacagtagtgatttcagcttggtgaaaacagacatggagagcaccgtggacaaatggctggatgtatcagagaaacttgaagaaaacatggataggctgagagtaagcctgtccatttgggatgatgtactgtcaactagagatgagattgagggatggtcaaacaactgcgttccacagatggcagaaaacatcagcaacctggataaccacctcagagctgaagaactgcttaaagaatttgagtctgaagttaaaaacaaagcattgagattggaagaactgcattccaaagttaatgatctgaaagaattaactaaaaatctagaaacaccgccagaccttcagtttatagaagcagacttaatgcagaaactggagcatgccaaagaaataactgaagtagcaaaaggaaccctgaaggatttcacggctcaaagtacacaagtggagaagtttattaatgacataacaacatggttcacaaaagtggaagaatcgttgatgaactgtgcccaaaatgagacttgtgaagcattgaaaaaagtcaaggatatacaaaaagaacttcaaagtcaacaaagcaacatcagctctacccaagaaaatctcaatagcttgtgccgcaagtaccactcagctgagttggagagcctgggccgtgcaatgactggtctgataaagaaacatgaagccgtgagccagttgtgctccaaaacccaggccagcctgcaggaatctctggaaaaacacttcagtgagtctatgcaggaattccaagaatggtttttgggagcaaaggcagcagcaaaagaatcatcagatcgcaccggtgacagcaaagttctagaagcaaagctccatgatcttcagaacattttggactcagtcagtgatgggcagagcaaacttgatgcagtgactcaagaaggacaaactttgtatgcacatttgtctaaacaaattgtcagtagcattcaagaacaaatcacaaaggccaatgaagagtttcaagcatttctgaaacaatgccttaaagataagcaggctcttcaagactgtgcttcagaacttggaagctttgaagatcagcacagaaaactgaacttatggatccatgaaatggaagaaaggttcaatacggaaaacttgggagagagtaaacagcacattcctgagaagaaaaatgaagttcataaagttgaaatgtttttgggagaactgctggctgcaagagagtctcttgataagctttcccagagagggcagcttctgagtgaagaaggccacggtgctgggcaggagggccgcctgtgttcccagctcctcacaagccaccagaacctacttagaatgaccaaagagaaactccggagctgccaggtggcccttcaggagcacgaagccctggaggaagcactgcaaagcatgtggttctgggtgaaggccattcaggacagactggcctgtgcagagagcactcttgggagcaaagacaccctggagaaacggctgtcacaaatacaggatattctcctgatgaaaggtgaaggggaagttaagttgaatatggccattggcaagggggaacaggccttgagaagtagcaacaaagaaggtcagagggtgattcagactcagttagagacccttaaagaagtgtgggctgacatcatgagctcctccgtccacgctcaaagcactttagagtctgtgattagccaatggaatgactatgtagagaggaaaaaccagttggagcagtggatggaatcagtggatcaaaaaatagaacatcccttacaaccacagccaggtctgaaagagaagttcgtcctgcttgaccacctccagtccatcctgtctgaggcagaagatcacacgagagcccttcaccgtctaattgcgaagtccagggagctctacgaaaagacagaggatgagtctttcaaggacacagctcaagaggagctgaaaacacagtttaatgatataatgactgttgccaaggaaaaaatgaggaaagtggaagagattgtgaaagatcatctaatgtatttagatgcggtccacgagttcacagattggctccattcagcaaaggaagaacttcaccggtggtcagatatgtctggagattcatcagccacccagaaaaagttatcaaaaattaaggagctgatagattccagagagattggtgcaagccgtctcagcagagtggagtcgctggctcccgaagtgaaacagaacacaactgccagtgggtgtgagctcatgcacacggagatgcaggccctgcgtgccgactggaagcagtgggaagacagtgtattccaaacgcagagctgtttggagaacctggtcagccagatggccctttcggagcaggaattctcaggccaagtggctcaactggagcaggccctggaacagttcagtgcccttctgaaaacctgggctcagcagttaaccctcctggaaggcaagaacacggatgaggagatagtggaatgctggcacaaaggacaagagatactggatgctttgcaaaaagcagagcctagaacagaggatctcaagtctcagctgaatgaactttgtcgattttccagagacctgagtacctacagtggaaaagtttctggcttgattaaagagtataattgtctttgtttgcaagcatccaagggctgccagaataaagaacagattttacagcaaagatttcgaaaggccttcagggatttccagcagtggttggttaatgcaaaaatcactaccgccaagtgttttgatatacctcaaaatataagtgaagtttcaactagtcttcagaaaatacaggagtttttgtcagaaagtgaaaatggacagcacaagctaaacatgatgctgtctaaaggggaacttctgagtaccctgctgaccaaagagaaagcgaaagggatccaggccaaagttacagctgcaaaagaagattggaaaaattttcattcaaatctccaccaaaaagaatctgctctagagaatctaaagatccaaatgaaggactttgaagtaag

SEQ ID NO: 10 - DNA, 1746 bp, homo sapiens
cgttcgctcgcttcgccttgctgctgggaaactgaacgaggccgagagagaaggttcttgagttcatgtaagaggacagtcttaaaacggaagaagaaaaagaagcagttcagtctttgggagagctgcctccttgttgagtgctgcaaaggcctggaattcatttatgacagaatagatctagaaaagtccaagcatgttttctagagtggtgtagccctgtgctgcctccagtgaagagtctcttggtgttggcttcgtgcttccggagggaccatggcaacctccagaggggcctcccggtgtcctcgggatatcgccaatgtgatgcagaggctgcaagatgagcaagagatagtacaaaaacgaactttcacaaaatggatcaactctcatctggccaagcggaaacctccaatggtggtggacgatctttttgaagacatgaaagatggtgttaaactgcttgcccttctggaggtcctgtctgggcagaaactgccttgtgaacaaggacgccggatgaagcgaatccatgctgtggctaacattggcacggcactcaagttcctcgaaggaagaaagattaaattagtcaacattaactccaccgatatagctgatggccgaccctcaatagttcttggattgatgtggaccattattctatatttccagattgaagagttgaccagcaacctgccccagctccagtctttgtccagcagcgcatcctccgtggacagcatagttagctctgagactcccagcccaccaagtaaacggaaggtgaccaccaagatccaaggaaatactaagaaggctttattaaagtgggttcagtacacagctggcaagcagactggaatagaagtaaaagattttgggaagagttggagaagcggggttgcctttcattcagttattcatgccattcgaccggaattggtggacttggagacagtgaaaggcagatccaaccgagaaaatttggaggatgctttcactatcgccgaaacagaactggggatcccaagactgctagatcctgaagacgttgatgtggataaaccagatgagaaatctattatgacctatgtagcccagtttctgaaacattatcctgacatccacaatgcaagcactgatgggcaagaggatgatgaaatacttccaggtttcccatcttttgcaaattctgtacaaaattttaagagagaagacggagtaatttttaaggaaatgaaagtttggatagaacaatttgagagagatttgacaagagcacagatggtggaatcaaatttacaggataaatatcagtcatttaagcacttcagagttcaatatgaaatgaagaggaaacagattgaacatttaatacaaccattacacagagacggtaaattgtcacttgaccaagcattggtaaaacaatcttgggatagagtgacctccaggctctttgactggcatatacagcttgataaatctcttcctgcacctctgggcaccataggtgcctggctgtacagagcggaggtggccctgagagaggaaataaccgttcaacaggtccacgaggaaacagcaaacacgatacaacggaaacttgagcaacataaggatctgcttcaaaacacggatgcccacaaaagagcattccatgaaatctaccggaccaggtctgttaacgggattccagtgccacctgatcaattagaggacatggccgagaggtttcat

SEQ ID NO: 11 - DNA, 53 bp, Homo sapiens
cgttcgctcgcttcgccttgctgctgggaaactgaacgaggccgagagagaag

SEQ ID NO: 12 - DNA, 1202 bp, Homo sapiens
gtcgacgatggcaacctccagaggggcctcccggtgtcctcgggatatcgccaatgtgatgcagaggctgcaagatgagcaagagatagtacaaaaacgaactttcacaaaatggatcaactctcatctggccaagcggaaacctccaatggtggtggacgatctttttgaagacatgaaagatggtgttaaactgcttgcccttctggaggtcctgtctgggcagaaactgccttgtgaacaaggacgccggatgaagcgaatccatgctgtggctaacattggcacggcactcaagttcctcgaaggaagaaagattaaattagtcaacattaactccaccgatatagctgatggccgaccctcaatagttcttggattgatgtggaccattattctatatttccagattgaagagttgaccagcaacctgccccagctccagtctttgtccagcagcgcatcctccgtggacagcatagttagctctgagactcccagcccaccaagtaaacggaaggtgaccaccaagatccaaggaaatgctaagaaggctttattaaagtgggttcagtacacagctggcaagcagactggaatagaagtaaaagattttgggaagagttggagaagcggggttgcctttcattcagttattcatgccattcgaccggaattggtggacttggagacagtgaaaggcagatccaaccgagaaaatttggaggatgctttcactatcgccgaaacagaactggggatcccaagactgctagatcctgaagacgttgatgtggataaaccagatgagaaatctattatgacctatgtagcccagtttctgaaacattatcctgacatccacaatgcaagcactgatgggcaagaggatgatagagaagacagagtaatttttaaggaaatgaaagtttggatagaacaatttgagagagatttgacaagagcacagatggtggaatcaaatttacaggataaatatcagtcatttaagcacttcagagttcaatatgaaatgaagaggaaacagattgaacatttaatacaaccattacacagagacggtaaattgtcacttgaccaagcattggtaaaacaatcttgggatagagtgacctccaggctctttgactggcatatacagcttgataaatctcttcctgcacctctgggcaccagggccc

SEQ ID NO: 13 - DNA, 21 bp, Homo sapiens
tccatgcacagaggatcaccg

SEQ ID NO: 14 - DNA, 51 bp, Homo sapiens
gaaatacttccaggtttcccatcttttgcaaattctgtacaaaattttaag

SEQ ID NO: 15 - DNA, 2461 bp, Homo sapiens
gtcgacagatttgacaagagcacagatggtggaatcaaatttacaggataaatatcagtcatttaagcacttcagagttcaatatgaaatgaagaggaaacagattgaacatttaatacaaccattacacagagacggtaaattgtcacttgaccaagcattggtaaaacaatcttgggatagagtgacctccaggctctttgactggcatatacagcttgataaatctcttcctgcacctctgggcaccataggtgcctggctgtacagagcggaggtggccctgagagaggaaataaccgttcaacaggtccacgaggaaacagcaaacacgatacaacggaaacttgagcaacataaggatctgcttcaaaacacggatgcccacaaaagagcattccatgaaatctaccggaccaggtctgttaacgggattccagtgccacctgatcaattagaggacatggccgagaggtttcattttgtttcctccacatcagagctacacctaatgaaaatggaatttttagaattaaagtaccgtctgctctcactgctggttcttgcagagtcaaagctgaagtcttggatcattaagtacgggaggagagagtcagtggagcagcttctacaaaactacgtgtcttttatagaaaatagcaagttctttgaacaatatgaggtgacataccagatcttgaaacagacagctgagatgtatgtcaaagcagatggttcagtggaagaagctgagaatgtgatgaaattcatgaatgaaaccaccgctcagtggaggaatctctcagtagaagtgaggagtgtgaggagcatgctggaagaagtgatctctaactgggatcgctatggcaatacagtggctagtctgcaagcctggctagaggatgctgaaaaaatgctcaatcaatcagaaaatgccaaaaaggatttttttcgaaatttacctcattggattcagcagcatactgccatgaacgatgctggcaattttctaattgaaacctgtgatgagatggtttcccgtgacctgaagcagcaattactgttgctaaatgggcggtggagggagttgtttatggaagtcaagcaatatgctcaagctgatgagatggacagaatgaagaaggaatacacagactgtgttgttaccctgtctgcttttgcaacggaagcccataagaaactttctgaacccttagaagtctcttttatgaatgtcaagctattaattcaagacttggaggatattgagcagagggtgcctgtgatggatgcccaatacaagataattacaaagacagcacacctcattgccaaagaaagcccccaagaagaaggaaaagaaatgtttgcgaccatgtcaaagctcaaagagcagctaaccaaggtcaaagaatgttactccccactcctttatgagtctcagcagctgttgattccgttggaggaattagaaaagcagatgacgtccttttatgactcacttgggaaaatcaatgaaattatcacagttcttgagcgtgaggcacaatcgagtgccctttttaaacaaaaacatcaggaactgttagcttgtcaagaaaactgtaagaaaaccttgacacttattgagaaaggcagtcaaagtgttcaaaagtttgtgaccttgagcaacgtgttaaagcattttgatcagacgaggctacaaagacagattgcagatattcatgttgcttttcagagtatggtaaagaaaactggagattggaagaagcatgtggaaaccaacagtcgcttgatgaagaagtttgaggagtctcgagcagagttggagaaggtactgcggattgctcaggagggcctggaggaaaagggggatccagaggagctcctgcggagacacactgagtttttcagtcagctggatcagagggtgctcaatgctttcctgaaagcttgtgatgaactcaccgacatccttccagagcaggagcagcaggggctgcaggaagctgttcgaaagctccacaaacaatggaaggatcttcaaggagaagccccttatcatttgcttcatctgaagattgatgtggagaagaataggttcttagcctctgcagaagaatgcagaactgagctggatcgagagaccaagctgatgccccaggaaggcagtgaaaagataattaaagagcacagggttttcttcagtgacaaaggtcctcatcatctctgtgagaaaaggttacagctcatcgaggaactctgtgtgaaactcccagtgcgggacccagtaagggacacacctggaacctgtcacgtgactctcaaagagctcagagctgccattgacagcacctacaggaagctcatggaagacccagacaagtggaaggactacactagcagattctccgagttctcatcttggatatctacaaatgagacacaattaaaggggccc

SEQ ID NO: 16 - DNA, 2551 bp, Homo sapiens
gtcgaccgaggaactctgtgtgaaactcccagtgggggacccagtaagggacacacctggaacctgtcacgtgactctcaaagagctcagagctgccattgacagcacctacaggaagctcatggaagacccagacaagtggaaggactacactagcagattctctgagttctcatcttggatatctacaaatgagacacaattaaaggggatcaagggtgaggccatcgatactgccaaccacggagaggttaaacgtgccgttgaagagatcagaaatggtgttaccaaaaggggtgagaccctcagctggctgaaatccaggctgaaagttttgacagaagtttcttctgagaatgaagcccaaaagcagggagatgagctggcaaaattatccagctctttcaaggctcttgtgacgctgctgtcagaggttgaaaagatgctaagcaattttggggactgtgtccagtacaaagaaatagtcaaaaattctctcgaagaattaatttctggctctaaagaagtccaggaacaagctgagaagatcttggatactgaaaatctgtttgaagcacagcagttacttcttcatcaccagcaaaagacaaagcggatcccagcaaagaagagagatgtgcagcagcagatcgcgcaggcgcagcagggagaaggggggctgcctgaccgaggccacgaggagctgcggaagctggagagctcactggatggcctggagcgcagccgggagaggcaggaacgccgcatccaggtcacattaagaaaatgggggcgatttgaaacaaacaaagaaacagtagtaagatacctttttcaaacaggttccagtcatgaacgcttcttgagttttagcagtttggaaagtttatcttcagaactggaacaaacaaaggagttttctaaacggacagaaagtattgcagtccaggctgagaaccttgtaaaggaagcttcagagataccgcttgggccccaaaataagcagctgcttcaacagcaggccaagtcaatcaaagaacaagtcaaaaaattagaagacacgcttgaagaagatattaaaaccatggaaatggtgaaaaccaagtgggatcattttggcagtaattttgagactctgtccgtctggataactgagaaagaaaaagaactcaatgccttggaaacttcgtcatctgccatggacatgcaaatcagccaaattaaggtcacaattcaggaaatagaaagtaagctcagcagcattgtaggattagaagaagaagcccagtcttttgctcagtttgttaccactggagaatctgctcgaattaaagccaagttgacacaaataagaagatacggggaagagcttcgagagcatgcacagtgtctggaaggaacaatcctgggacatttatctcagcagcaaaagtttgaagagaaccttagaaagatccagcaatctgtgtctgaatttgaagataaacttgctgttccaattaaaatatgttcttcagctacagaaacatacaaagttcttcaagaacatatggatctctgccaggccctggagtcactgagcagcgcgatcactgccttctcagccagtgccaggaaggttgtgaacagagattcctgtgttcaggaggctgcggctctacagcagcaatacgaggacatcctaaggagggcgaaggagagacagacggcgctggagaatctgctggcccactggcagaggctagagaaagaactatcatcctttttgacctggttagagcggggtgaagctaaagccagttccccagaaatggacatttctgcagacagagtcaaagtggaaggtgaacttcagttaatacaggcactgcaaaatgaagttgtatcccaggcctcattctatagcaaacttttgcaattgaaggaatcattgttctcagtagcctccaaagatgatgtgaaaatgatgaaactacatttggagcagttggatgagagatggagagatttaccacagatcattaacaaaaggattaattttcttcagtctgtggttgctgaacaccagcaatttgatgagctgctgctttccttttctgtctggattaaactgtttcttagtgaattacaaactacctctgagattagcataatggaccatcaagtagcccttactcggcataaggaccacgcagcagaagtagagagcaaaaagggcgaattgcagagtctgcagggtcacttagcaaagttgggttctctgggccgtgctgaggacctccacctcctgcagggaaaggctgaggactgcttccagctgtttgaggaggccagccgggttgtggagaggcggcagcttgccctgtcccatttggcagaattcctccagagccatgcctctctgtccggcattctccgccagctgaggcaaacagtggaagcaaccaacagtatgaataagaacgagtctgatttgatagaaaaggacctcaatgatgctcttcaaaatgctaaagcattagaatctgctgccgtcagtctggatgggccc

SEQ ID NO: 17 - DNA, 1120 bp, Homo sapiens
ggagggcgaaggagagacagacggcgctggagaatctgctggcccactggcagaggctagagaaagaactatcatcctttttgacctggttagagcggggtgaagctaaagccagttccccagaaatggacatttctgcagacagagtcaaagtggaaggtgaacttcagttaatacaggcactgcaaaatgaagttgtatcccaggcctcattctatagcaaacttttgcaattgaaggaatcattgttctcagtagcctccaaagatgatgtgaaaatgatgaaactacatttggagcagttggatgagagatggagagatttaccacagatcattaacaaaaggattaattttcttcagtctgtggttgctgaacaccagcaatttgatgagctgctgctttccttttctgtctggattaaactgtttctcagtgaattacaaactacctctgagattagcataatggaccatcaagtagcccttactcggcataaggaccacgcagcagaagtagagagcaaaaagggcgaattgcagagtctgcagggtcacttagcaaagttgggttctctgggccgtgctgaggacctccacctcctgcagggaaaggctgaggactgcttccagctgtttgaggaggccagccaggttgtggagaggcggcagcttgccctgtcccatttggcagaattcctccagagccatgcctctctgtccggcattctccgccagctgaggcaaacagtggaagcaaccaacagtatgaataagaacgagtctgatttgatagaaaaggacctcaatgatgctcttcaaaatgctaaagcattagaatctgctgccgtcagtctggatggtattctttccaaagcccaataccatctgaaaatcgggagctctgagcaaaggacttcctgcagagccacggctgatcagctctgtggagaggtagagaggatccagaaccttctgggaaccaagcagagtgaggcagatgctctggcagtgttgaaaaaagcattccaagaccagaaagaggagcttctgaaaagcattgaggacattgaagaaaggactgacaaagagcgattgaaagaacctacccgccaagctct

SEQ ID NO: 18 - DNA, 1601 bp, Homo Sapiens
cattctttccaaagcccaataccatctgaaaatcgggagctctgagcaaaggacttcctgcagagccacggctgatcagctctgtggagaggtagagaggatccagaaccttctgggaaccaagcagagtgaggcagatgctctggcagtgttgaaaaaagcattccaagaccagaaagaggagcttctgaaaagcattgaggacattgaagaaaggactgacaaagagcgattgaaagaacctacccgccaagctcttcagcagaggttaagagtgtttaatcagctagaagatgaattgaattctcacgagcatgaactatgttggttgaaagacaaagccaagcaaattgcccagaaagatgtagcttttgcacctgaagttgacagggagataaaccgcttagaggtcacctgggatgataccaaaagactaattcatgaaaatcagggtcagtgctgtggacttattgacttaatgagagaatatcagaacctgaaatcagctgtatctaaagtcttagaaaatgccagcagtgtgattgtaaccagaactaccataaaagatcaggaggatcttaaatgggctttttccaagcatgaaactgccaagaacaaaatgaattacaaacagaaagacttggataactttaccagcaaaggaaaacacttgttatctgagctgaagaaaattcacagtagtgatttcagcttggtgaaaacagacatggagagcaccgtggacaaatggctggatgtatcagagaaacttgaagaaaacatggataggctgagagtaagcctgtccatttggggtgatgtactgtcaactagagatgagattgagggatggtcaaacaactgcgttccacagatggcagaaaacatcagcaacctggataaccacctcagagctgaagaactgcttaaagaatttgagtctgaagttaaaaacaaagcattgagattggaagaactgcattccaaagttaatgatctgaaagaattaactaaaaatctagaaacaccgccagaccttcagtttatagaagcagacttaatgcagaaactggagcatgccaaagaaataactgaagtagcaaaaggaaccctgaaggatttcacggctcaaagtacacaagtggagaagtttattaatgacataacaacatggttcacaaaagtggaagaatcgttgatgaactgtgcccaaaatgagacttgtgaagcattgaaaaaagtcaaggatatacaaaaagaacttcaaagtcaacaaagcaacatcagctctacccaagaaaatctcaatagcttgtgccgcaagtaccactcagctgagttggagagcctgggccgtgcaatgactggtctgataaagaaacatgaagccgtgagccagttgtgctccaaaacccaggccagcctgcaggaatctctggaaaaacacttcagtgagtctatgcaggaattccaagaatggtttttgggagcaaaggcagcagcaaaagaatcatcagatcgcaccggtgacagcaaagttctagaagcaaagctccatgatcttcagaacattttggactcagtcagtgatgggcagagca

SEQ ID NO: 19 - DNA, 1694 bp, Homo sapiens
gccgtgcaatgcctggtctgataaagaaacatgaagccgtgagccagttgtgctccaaaacccaggccagcctgcaggaatctctggaaaaacacttcagtgagtctatgcaggaattccaagaatggtttttgggagcaaaggcagcagcaaaagaatcatcagatcgcaccggtgacagcaaagttctagaagcaaagctccatgatcttcagaacattttggactcagtcagtgatgggcagagcaaacttgatgcagtgactcaagaaggacaaactttgtatgcacatttgtctaaacaaattgtcagtagcattcaagaacaaatcacaaaggccaatgaagagtttcaagcatttctgaaacaatgccttaaagataagcaggctcttcaagactgtgcttcagaacttggaagctttgaagatcagcacagaaaactgaacttatggatccatgaaatggaagaaaggttcaatacggaaaacttgggagagagtaaacagcacattcctgagaagaaaaatgaagttcataaagttgaaatgtttttgggagaactgctggctgcaagagagtctcttgataagctttcccagagagggcagcttctgagtgaagaaggccacggtgctgggcaggagggccgcctgtgttcccagctcctcacaagccaccagaacctacttagaatgaccaaagagaaactccggagctgccaggtggcccttcaggagcacgaagccctggaggaagcactgcaaagcatgtggttctgggtgaaggccattcaggacagactggcctgtgcagagagcactcttgggagcaaagacaccctggagaaacggctgtcacaaatacaggatattctcctgatgaaaggtgaaggggaagttaagttgaatatggccattggcaagggggaacaggccttgagaagtagcaacaaagaaggtcagagggtgattcagactcagttagagacccttaaagaagtgtgggctgacatcatgagctcctccgtccacgctcaaagcactttagagtctgtgattagccaatggaatgactatgtagagaggaaaaaccagttggagcagtggatggaatcagtggatcaaaaaatagaacatccctcacaaccacagccaggtctgaaagagaagttcgtcctgcttgaccacctccagtccatcctgtctgaggcagaagatcacacgagagcccttcaccgtctaattgcgaagtccagggagttctacgaaaagacagaggatgagtctttcaaggacacagctcaagaggagctgaaaacacagtttaatgatataatgactgttgccaaggaaaaaatgaggaaagtggaagagattgtgaaagatcatctaatgtatttagatgcggtccacgagttcacagattggctccattcagcaaaggaagaacttcaccggtggtcagatatgtctggagattcatcagccacccagaaaaagttatcaaaaattaaggagctgatagattccagagagattggtgcaagccgtctcagcagagtggagtcgctggctcccgaagtgaaacagaacacaactgccagtgggtgtgagctcatgcacacggagatgcaggccctgcgtgccgactggaagcagtgggaagacagtgtattccaaacgcagagctgt

SEQ ID NO: 20 - DNA, 1505 bp, Homo sapiens
gtcgactctcctcacaagccaccagaacctacttagaatgaccaaagagaaactccggagctgccaggtggcccttcaggagcacgaagccctggaggaagcactgcaaagcatgtggttctgggtgaaggccattcaggacagactggcctgtgcagagagcactcttgggagcaaagacaccctggagaaacggctgtcacaaatacaggatattctcctgatgaaaggtggaggggaagttaagttgaatatggccattggcaagggggaacaggccttgagaagtagcaacaaagaaggtcagagggtgattcagactcagttagagacccttaaagaagtgtgggctgacatcatgagctcctccgtccacgctcaaagcactttagagtctgtgattagccaatggaatgactatgtagagaggaaaaaccagttggagcagtggatggaatcagtggatcaaaaaatagaacatcccttacaaccacagccaggtctgaaagagaagttcgtcctgcttgaccacctccagtccatcctgtctgaggcagaagatcacacgagagcccttcaccgtctaattgtgaagtccagggagctctacgaaaagacagaggatgagtctttcaaggacacagctcaagaggagctgaaaacacagtttaatgatataatgactgttgccaaggaaaaaatgaggaaagtggaagagattgtgaaagatcatctaatgtatttagatgcggtccacgagttcacagattggctccattcagcaaaggaagaacttcaccggtggtcagatatgtctggagattcatcagccacccagaaaaagttatcaaaaattaaggagctgatagattccagagagattggtgcaagccgtctcagcagagtggagtcgctggctcccgaagtgaaacagaacacaactgccagtgggtgtgagctcatgcacacggagatgcaggccctgcgtgccgactggaagcagtgggaagacagtgtattccaaacgcagagctgtttggagaacctggtcagccagatggccctttcggagcaggaattctcaggccaagtggctcaactggagcaggccctggaacagttcagtgcccttctgaaaacctgggctcagcagttaaccctcctggaaggcaagaacacggatgaggagatagtggaatgctggcacaaaggacaagagatactggatgctttgcaaaaagcagagcctagaacagaggatctcaagtctcagctgaatgaactttgtcgattttccagagacctgagtacctacagtggaaaagtttctggcttgattaaagagtataattggtgagcatgaaccttattggtgttcaagatatttttatacaaattagaagctagaagtttattttaaggtgtaatttcaaaataatattagttcagcattaaatatattactaagtgaggtaagtgcacattctaagaactggtgacccggg

SEQ ID NO: 21 - DNA, 681 bp, Homo sapiens
acaaggcaccagaacctacttagaatgaccaaagagaaactccggagctgccaggtggcccttcaggagcacgaagccctggaggaagcactgcaaagcatgtggttctgggtgaaggccattcaggacagactggcctgtgcagagagcactcttgggagcaaagacaccctggagaaacggctgtcacaaatacaggatattctcctgatgaaaggtgaaggggaagttaagttgaatatggccattggcaagggggaacaggccttgagaagtagcaacaaagaaggtcagagggtgattcagactcagttagagacccttaaagaagtgtgggctgacatcatgagctcctccgtccacgctcaaagcactttagagtctgtgattagccaatggaatgactatgtagagaggaaaaaccagttggagcagtggatggaatcagtggatcaaaaaatagaacatcccttacaaccacagccaggtctgaaagagaagttcgtcctgcttgaccacctccagtccatcctgtctgaggcagaagatcacacgagagcccttcaccgtctaattgcgaagtccagggagctctacgaaaagacagaggatgagtctttcaaggacacagctcaagaggagctgaaaacacagtttaatgatataatgactgttgccaaa

SEQ ID NO: 22 - DNA, 24 bp, Homo sapiens
ctggagaaacggctgtcacaaata

SEQ ID NO: 23 - DNA, 28 bp, Homo sapiens
tagccaatggaatgactatgtagagagg

SEQ ID NO: 24 - DNA, 1093 bp, Homo sapiens
atgtatttagatgcggtccacgagttcacagattggctccattcagcaaaggaagaacttcaccggtggtcagatatgtctggagattcatcagccacccagaaaaagttatcaaaaattaaggagctgatagattccagagagattggtgcaagccgtctcagcagagtggagtcgctggctcccgaagtgaaacagaacacaactgccagtgggtgtgagctcatgcacacggagatgcaggccctgcgtgccgactggaagcagtgggaagacagtgtattccaaacgcagagctgtttggagaacctggtcagccagatggccctttcggagcaggaattctcaggccaagtggctcaactggagcaggccctggaacagttcagtgcccttctgaaaacctgggctcagcagttaaccctcctggaaggcaagaacacggatgaggagatagtggaatgctggcacaaaggacaagagatactggatgctttgcaaaaagcagagcctagaacagaggatctcaagtctcagctgaatgaactttgtcgattttccagagacctgagtacctacagtggaaaagtttctggcttgattaaagagtataattggtgagcatgaaccttattggtgttcaagatatttttatacaaattagaagctagaagtttattttaaggtgtaatttcaaaataatattagttcagcattaaatatattactaagtgaggtaagtgcacattctaagaactggtgattctatcaaacatgattttcatcaactttatcgctttttcctgccattaaatttttgcgtcaccttttcagtctttgtttgcaagcatccaagggctgccagaataaagaacagattttacagcaaagatttcgaaaggccttcagggatttccagcagtggttggttaatgcaaaaatcactaccgccaagtgttttgatatacctcaaaatataagtgaagtttcaactagtcttcagaaaatacaggtaagggtgttatcaattaattctagtagcagtgctgatgtgaactaaagttaacctctaaatcaagggtccccagtcgac

SEQ ID NO: 25 - DNA, 357 bp, Homo sapiens
gagctgatagattccagagagattggtgcaagccgtctcagcagagtggagtcgctggctcccgaagtgaaacagaacacaactgccagtgggtgtgagctcatgcacacggagatgcaggccctgcgtgccgactggaagcagtgggaagacagtgtattccaaacgcagagctgtttggagaacctggtcagccagatggccctttcggagcaggaattctcaggccaagtggctcaactggagcaggccctggaacagttcagtgcccttctgaaaacctgggctcagcagttaaccctcctggaaggcaagaacacggatgaggagatagtggaatgctggcacaaaggacaa

SEQ ID NO: 26 - DNA, 137 bp, Homo sapiens
gagatactggatgctttgcaaaaagcagagcctagaacagaggatctcaagtctcagctgaatgaactttgtcgattttccagagacctgagtacctacagtggaaaagtttctggcttgattaaagagtataattg

SEQ ID NO: 27 - DNA, 878 bp, Homo sapiens
ggagatagtggaatgctggcacaaaggacaagagatactggatgctttgcaaaaagcagagcctagaacagaggatctcaagtctcagctgaatgaactttgtcgattttccagagacctgagtacctacagtggaaaagtttctggcttgattaaagagtataattggtgagcatgaaccttattggtgttcaagatatttttatacaaattagaagctagaagtttattttaaggtgtaatttcaaaataatattagttcagcattaaatatattactaagtgaggtaagtgcacattctaagaactggtgattctatcaaacatgattttcatcaactttatcgctttttcctgccattaaatttttgcgtcaccttttcagtctttgtttgcaagcatccaagggctgccagaataaagaacagattttacagcaaagatttcgaaaggccttcagggatttccagcagtggttggttaatgcaaaaatcactaccgccaagtgttttgatatacctcaaaatataagtgaagtttcaactagtcttcagaaaatacaggtaagggtgttatcaattaattctagtagcagtgctgatgtgaactaaagttaacctctaaatcaagggtccccatctcctgggccgcggactggtacaggtccctggcctgttaggaaccaggccgcacagcaggaggtgagcagcaggccaatgagcgttactgcctgagctccacctcctgtcagatcagcggtagcatcagattctcataggagcctgaaccccattgtgaactgtgcacacagaggaactaggctgtgtgctccttctaagaatctaactaatgcctgatgatcgaggtagaacagtttc

SEQ ID NO: 28 - DNA, 723 bp, Homo sapiens
gagatactggatgctttgcaaaaagcagagcctagaacagaggatctcaagtctcagctgaatgaactttgtcgattttccagagacctgagtacctacagtggaaaagtttctggcttgattaaagagtataattggtgagcatgaaccttattggtgttcaagatatttttatacaaattagaagctagaagtttattttaaggtgtaatttcaaaataatattagttcagcattaaatatattactaagtgaggtaagtgcacattctaagaactggtgattctatcaaacatgattttcatcaactttatcgctttttcctgccattaaatttttgcgtcaccttttcagtctttgtttgcaagcatccaagggctgccagaataaagaacagattttacagcaaagatttcgaaaggccttcagggatttccagcagtggttggttaatgcaaaaatcactaccgccaagtgttttgatatacctcaaaatataagtgaagtttcaactagtcttcagaaaatacaggtaagggtgttatcaattaattctagtagcagtgctgatgtgaactaaagttaacctctaaatcaagggtccccatctcctgggccgcggactggtacaggtccctggcctgttaggaaccaggccgcacagcaggaggtgagcagcaggccaatgagcgttactgcctgagctccacctcctgtcagatc

SEQ ID NO: 29 - DNA, 178 bp, Homo sapiens
tctttgtttgcaagcatccaagggctgccagaataaagaacagattttacagcaaagatttcgaaaggccttcagggatttccagcagtggttggttaatgcaaaaatcactaccgccaagtgttttgatatacctcaaaatataagtgaagtttcaactagtcttcagaaaatacag

SEQ ID NO: 30 - protein, 3032 aa, Homo sapiens
MATSRGASRCPRDIANVMQRLQDEQEIVQKRTFTKWINSHLAKRKPPMVVDDLFEDMKDGVKLLALLEVLSGQKLPCEQGRRMKRIHAVANIGTALKFLEGRKIKLVNINSTDIADGRPSIVLGLMWTIILYFQIEELTSNLPQLQSLSSSASSVDSIVSSETPSPPSKRKVTTKIQGNAKKALLKWVQYTAGKQTGIEVKDFGKSWRSGVAFHSVIHAIRPELVDLETVKGRSNRENLEDAFTIAETELGIPRLLDPEDVDVDKPDEKSIMTYVAQFLKHYPDIHNASTDGQEDDREDRVIFKEMKVWIEQFERDLTRAQMVESNLQDKYQSFKHFRVQYEMKRKQIEHLIQPLHRDGKLSLDQALVKQSWDRVTSRLFDWHIQLDKSLPAPLGTIGAWLYRAEVALREEITVQQVHEETANTIQRKLEQHKDLLQNTDAHKRAFHEIYRTRSVNGIPVPPDQLEDMAERFHFVSSTSELHLMKMEFLELKYRLLSLLVLAESKLKSWIIKYGRRESVEQLLQNYVSFIENSKFFEQYEVTYQILKQTAEMYVKADGSVEEAENVMKFMNETTAQWRNLSVEVRSVRSMLEEVISNWDRYGNTVASLQAWLEDAEKMLNQSENAKKDFFRNLPHWIQQHTAMNDAGNFLIETCDEMVSRDLKQQLLLLNGRWRELFMEVKQYAQADEMDRMKKEYTDCVVTLSAFATEAHKKLSEPLEVSFMNVKLLIQDLEDIEQRVPVMDAQYKIITKTAHLITKESPQEEGKEMFATMSKLKEQLTKVKECYSPLLYESQQLLIPLEELEKQMTSFYDSLGKINEIITVLEREAQSSALFKQKHQELLACQENCKKTLTLIEKGSQSVQKFVTLSNVLKHFDQTRLQRQIADIHVAFQSMVKKTGDWKKHVETNSRLMKKFEESRAELEKVLRIAQEGLEEKGDPEELLRRHTEFFSQLDQRVLNAFLKACDELTDILPEQEQQGLQEAVRKLHKQWKDLQGEAPYHLLHLKIDVEKNRFLASVEECRTELDRETKLMPQEGSEKIIKEHRVFFSDKGPHHLCEKRLQLIEELCVKLPVRDPVRDTPGTCHVTLKELRAAIDSTYRKLMEDPDKWKDYTSRFSEFSSWISTNETQLKGIKGEAIDTANHGEVKRAVEEIRNGVTKRGETLSWLKSRLKVLTEVSSENEAQKQGDELAKLSSSFKALVTLLSEVEKMLSNFGDCVQYKEIVKNSLEELISGSKEVQEQAEKILDTENLFEAQQLLLHHQQKTKRISAKKRDVQQQIAQAQQGEGGLPDRGHEELRKLESTLDGLERSRERQERRIQVTLRKWERFETNKETVVRYLFQTGSSHERFLSFSSLESLSSELEQTKEFSKRTESIAVQAENLVKEASEIPLGPQNKQLLQQQAKSIKEQVKKLEDTLEEDIKTMEMVKTKWDHFGSNFETLSVWITEKEKELNALETSSSAMDMQISQIKVTIQEIESKLSSIVGLEEEAQSFAQFVTTGESARIKAKLTQIRRYGEELREHAQCLEGTILGHLSQQQKFEENLRKIQQSVSEFEDKLAVPIKICSSATETYKVLQEHMDLCQALESLSSAITAFSASARKVVNRDSCVQEAAALQQQYEDILRRAKERQTALENLLAHWQRLEKELSSFLTWLERGEAKASSPEMDISADRVKVEGELQLIQALQNEVVSQASFYSKLLQLKESLFSVASKDDVKMMKLHLEQLDERWRDLPQIINKRINFLQSVVAEHQQFDELLLSFSVWIKLFLSELQTTSEISIMDHQVALTRHKDHAAEVESKKGELQSLQGHLAKLGSLGRAEDLHLLQGKAEDCFQLFEEASQVVERRQLALSHLAEFLQSHASLSGILRQLRQTVEATNSMNKNESDLIEKDLNDALQNAKALESAAVSLDGILSKAQYHLKIGSSEQRTSCRATADQLCGEVERIQNLLGTKQSEADALAVLKKAFQDQKEELLKSIEDIEERTDKERLKEPTRQALQQRLRVFNQLEDELNSHEHELCWLKDKAKQIAQKDVAFAPEVDREINRLEVTWDDTKRLIHENQGQCCGLIDLMREYQNLKSAVSKVLENASSVIVTRTTIKDQEDLKWAFSKHETAKNKMNYKQKDLDNFTSKGKHLLSELKKIHSSDFSLVKTDMESTVDKWLDVSEKLEENMDRLRVSLSIWDDVLSTRDEIEGWSNNCVPQMAENISNLDNHLRAEELLKEFESEVKNKALRLEELHSKVNDLKELTKNLETPPDLQFIEADLMQKLEHAKEITEVAKGTLKDFTAQSTQVEKFINDITTWFTKVEESLMNCAQNETCEALKKVKDIQKELQSQQSNISSTQENLNSLCRKYHSAELESLGRAMTGLIKKHEAVSQLCSKTQASLQESLEKHFSESMQEFQEWFLGAKAAAKESSDRTGDSKVLEAKLHDLQNILDSVSDGQSKLDAVTQEGQTLYAHLSKQIVSSIQEQITKANEEFQAFLKQCLKDKQALQDCASELGSFEDQHRKLNLWIHEMEERFNTENLGESKQHIPEKKNEVHKVEMFLGELLAARESLDKLSQRGQLLSEEGHGAGQEGRLCSQLLTSHQNLLRMTKEKLRSCQVALQEHEALEEALQSMWFWVKAIQDRLACAESTLGSKDTLEKRLSQIQDILLMKGEGEVKLNMAIGKGEQALRSSNKEGQRVIQTQLETLKEVWADIMSSSVHAQSTLESVISQWNDYVERKNQLEQWMESVDQKIEHPLQPQPGLKEKFVLLDHLQSILSEAEDHTRALHRLIAKSRELYEKTEDESFKDTAQEELKTQFNDIMTVAKEKMRKVEEIVKDHLMYLDAVHEFTDWLHSAKEELHRWSDMSGDSSATQKKLSKIKELIDSREIGASRLSRVESLAPEVKQNTTASGCELMHTEMQALRADWKQWEDSVFQTQSCLENLVSQMALSEQEFSGQVAQLEQALEQFSALLKTWAQQLTLLEGKNTDEEIVECWHKGQEILDALQKAEPRTEDLKSQLNELCRFSRDLSTYSGKVSGLIKEYNW

(19) United States
(12) Patent Application Publication
(10) Pub. No.: US 2007/0093436 A1
(43) Pub. Date: Apr. 26, 2007

(54) Drop1, a novel marker for carcinogenesis

(75) Inventors: Herwig Ponstingl, Heidelberg (DE); Hans-Peter Zimmermann, Heidelberg (DE); Gerhard Moldenhauer, Heidelberg (DE); Alexander Marme, Heidelberg (DE); Gunther Bastert, Heidelberg (DE); Raffael Kurek, Tubingen (DE); Dietheim Wallwiener, Tubingen (DE)

Correspondence Address: WILMER CUTLER PICKERING HALE AND DORR LLP, 399 PARK AVENUE, NEW YORK, NY, 10022, US

(73) Assignees: Deutsches Kresforschungszentrum Stiftung Des Offentlichen Rechts, Heidelberg (DE); Ruprecht-Karls- Universitat Heidelberg, Heidelberg (DE)

(21) Appl. No.: 10/551,878

(22) PCT Filed: Apr. 2, 2004
(86) PCT No.: PCT/EP04/03538

§ 371 Date: Nov. 14, 2006

(30) Foreign Application Priority Data
Apr. 3, 2003 (EP) 03007680.6

(51) Int. Cl. A61K 48/00; C12Q 1/68; G01N 33/53; C07H 21/04

(52) U.S. Cl. 514/44; 435/6; 435/7.1; 435/69.1; 435/320.1; 435/325; 530/350; 536/23.5

(57) ABSTRACT

Described is a novel marker protein associated with epithelial tumors, e.g., ovarian, mammary, stomach, kidney, thyroid and lung carcinomas, Dropl, a dystrophin-related protein as well as a nucleic acid molecule encoding Dropl. Furthermore, various diagnostic and therapeutic uses based on the finding that the expression of Dropl is downregulated in epithelial tumors are described.

The present invention relates to gene expression in normal cells and cells of tumors and particularly to a novel marker protein associated with epithelial tumors, e.g., ovarian, mammary, stomach, kidney, thyroid, and lung carcinomas, Drop1, a dystrophin-related protein as well as a nucleic acid molecule encoding Drop1. Furthermore, the present invention relates to various diagnostic and therapeutic uses based on the finding that the expression of Drop1 is downregulated in epithelial tumors.

Tumorigenesis represents a complex multistage process in which genetic changes and environmental factors are thought to deregulate the cellular processes that control cell proliferation and differentiation. Among the gynaecological tumors, ovarian carcinoma has the highest mortality. It typically metastasizes to the peritoneal cavity. A comprehensive biological characterisation is crucial to improve the prognosis for cancer, e.g., ovarian cancer patients. Though morphological characterisation of the tumor in many cases gives a good hint towards prognosis and prediction of the response to specific therapies, its accuracy is limited and fails in many cases. Especially some tumors of low malignant potential turn out to be much more dangerous than initially thought. Therefore, markers are important which define the malignancy by its biological characteristics on the level of gene expression. These markers can also predict the possible success of a chemotherapy. Im mammary carcinoma, for instance, tumors overexpressing HER2/neu respond better to chemotherapy involving a taxane and anthracyclin than to one using alkylating agents. Nuclear proteins interacting with chemotherapeutic agents might also be involved in the cytotoxic mechanisms of these drugs, or their modified expression might be responsible for the failure of the treatment. In summary, the use of reliable diagnostic molecular markers would be helpful for an understanding of the molecular basis of tumors, e.g. ovarian and mammary carcinomas, for distinguishing benign from malign tissue and for grading and staging carcinoma. It can be expected that such markers are also useful for the development of novel therapeutic avenues for cancer treatment.

Thus, the technical problem underlying the present invention is to provide means for diagnosis and therapy of tumors, in particular epithelial carcinomas, which overcome the disadvantages of the prior art diagnostic and therapeutic methods.

The solution to said technical problem is achieved by providing the embodiments characterized in the claims.

During the experiments leading to the present invention a gene encoding a novel protein, Drop1, was found, which is downregulated in most cases of several types of carcinoma. Its expression is downregulated 14- to 210-fold in 85% of the ovarian and mammary carcinomas investigated. Similar downregulation was observed in a cancer profiling array for uterine, stomach, kidney, lung, and thyroid carcinomas. The Drop1 mRNA is present in only two out of eight ovarian carcinoma cell lines. The Drop1 gene comprises 56 exons in a genomic sequence of 180 kb. Starting from a differential display fragment of 680 bp, the 10.5 kb mRNA sequence was completed using RACE and an ovarian cDNA library as a template. The results were confirmed by overlapping PCR products using total cDNA from ovarian epithelial cells. Northern blots also show a diffuse band at 10-11 kb, mainly expressed in testes and in ovary. This was confirmed by in situ hybridisation. The gene is located in the chromosome region 6q25, where a loss of heterozygosity has been described for several types of carcinomas. Therefore, deletions may also cause down-regulation of this gene. The ORF predicts a protein of 349 kDa with a pI of 5.7, the sequence of which is only distantly related to muscle dystrophin, yet the overall structure is similar. It features two calponin homology domains and 12 spectrin-like repeats. Such domains are also found in muscle dystrophin. Their loss or truncation results in progressive muscular dystrophy with frequently fatal consequences. Drop1, in addition, displays an AAA domain of some 220 amino acid residues, which characterizes a large family of ATPases acting as ATP-dependent protein clamps. Dystrophin is present at the cytoplasmic membrane, where it integrates proteins into sterically fixed complexes. Drop1, in contrast, is located at or in the nucleus, where it may have a similar integrating function for nuclear protein complexes. Drop1 malfunction may impede cell cycle regulation, centrosome orientation and chromosome distribution. Dysfunction of mechanisms regulating these processes occur during pathogenesis and are key problems in malignant disease.

During the experiments resulting in the present invention it has been shown that Drop1 has high homology in an overlap to recently published proteins with unknown functions called enaptin (EMBL/Genbank/DDBJ databases AF535142), Nesprin-1 (Zhang et al., Genomics 80(5), pp. 473-481 (2002)), KIAA1756 (EMBL/Genbank/DDBJ databases AB051543) and Syne-1 (EMBL/Genbank/DDBJ databases AY135172). Furthermore, some sequences having homologies to Drop1 are shown in WO-A-02/40715. However, this international publication comprises a huge number of sequences without any specific relation to a disease. It is not plausible that all these sequences play the same or any role in such a large number of mentioned diseases.

In summary, Drop1 is a novel gene downregulated in epithelial tumors with a broad range of characteristics relevant for tumor biology. Downregulation, e.g. by promotor methylation, deletion, or truncation is expected to contribute to the development of carcinomas. Thus, Drop1 is very promising as a prognostic as well as a predictive marker.

### BRIEF DESCRIPTION OF THE DRAWINGS

FIG. 1: Differential display with cDNA from normal and tumor tissue of patient 31, using anchor primer G and arbitrary primer DOC, generated a cDNA fragment 31N/GDOC/1 dd (arrow) To avoid artefacts, RNA from both sources was twice reversely transcribed, and two differential display experiments were performed with each of the resulting four cDNAs, yielding eight samples run in parallel on the same gel for comparison. The figure shows the autoradiogram of the bands. The differentially expressed band was excised from the gel, eluted, amplified and cloned into pCRII-TOPO.

FIG. 2: Mutitissue Northern Blot hybridized with a randomly promed probe using the insert of clone 3R9 (see FIG. 6) as a template

FIG. 3: Autoradiogram of a cancer profiling array (BD Biosciences Clontech, Cat.# 7841-1 Lot #2040887) including amplified and normalized cDNA from 241 tumor (C) and corresponding normal tissues (N) from individual patients, spotted side by side Boxed areas contain three cDNA spots from the same patient: top left normal, top right metastatic tissue, bottom right primary tumor. The array had previously been tested for equal loading of cDNA in the spots by hybridisation with a digoxigenin-labeled cDNA probe. Clone 3R9 was used to generate the radioactive Drop1 probe for hybridisation.

FIG. 3a: Autoradiogram of a cancer profiling array II (BD Biosciences Clontech, Cat#S2555; Lot # 2060252) hybridized with 3R9 as in FIG. 3. This array includes normalized cDNA from 154 tumor and corresponding normal tissues from individual patients, amplified using BD SMART technology. cDNA from nine cancer cell lines is included at the lower right. In addition to supporting the data obtained with cancer profiling array I these results extend the observation of downregulated Drop1 to cervix, vulva, and pancreas carcinomas.

FIG. 4: Comparative in situ hybridisation of tissue sections with an antisense probe generated from clone 3R9 Right: both, tube epithelium (top) and ovarian surface epithelium (bottom) of patient 91 express Drop1 at a comparable level. Left: Tube epithelium (top) of patient 87 expresses Drop1 at a considerably higher level that the ovarian carcinoma (bottom) of the same patient.

FIG. 5: Chromosome region 6q25.1, containing the Drop1 gene Several groups have reported loss of heterozygosity for this region in ovarian, breast, uterine, and non small cell lung cancer, as indicated. Top line: chromosomal markers. Estrogen receptor 1 (ESR1, alpha) and early mitotic inhibitor 1(EMI1) are expressed from the plus strand (>), Drop1 from the minus strand (<).

FIG. 6: Sequence details for Drop1 The figure shows the location in the contig of sequences mentioned in the text (A). The sequence was established starting with the 681 bp of the differential display fragment 31N/GDOC/1 dd. Primers L467 and U285 (their direction inverted in the final ORF) were derived from it and used throughout for quantitative PCR. “RACE walking”experiments were performed, based on a commercial RACE ovarian library of these, 5R9 marks the 5′ region of drop1, 3R9 was used to generate the probes for in situ hybridisation, for multitissue Northern blots, and for the cancer profiling array. For validation of these sequences by overlapping PCR, total cDNA from SKOV-3 ovarian carcinoma cells was used as a template (Drop 1A,C,B,D). The same template yielded the clones used in bacterial and mammalian vectors for expression of domains (Drop1 alpha, beta, gamma, epsilon, and C-terminal domain). In total, more than 70 clones were aligned. Only upon completion of the Drop1 structure the enaptin sequence became available, of which here the 5′ 10 000 bp are shown (B). For ease of comparison the exons are designated accordingly. Drop1 is encoded by the 5′ region (exons 2 to 58) of the enaptin gene (147 exons). The Drop1 mRNA and protein, however, are entities different from enaptin. Enaptin exons 07 and 12 are not used in Drop1, and enaptin-exon 58 is read differently: for Drop1 it is read beyond the enaptin-exon 58-intron transition. It carries the stop codon for the ORF and a long 3′ UTR that includes the unused sequence of enaptin-exon 59.

FIG. 7: Drop1 protein domains and overlapping expression clones CH1, 2: calponin homology domains, AAA: ATPase domain, NLS: putative nuclear localization signal, S1 to S12: spectrin-like domains alpha to epsilon: overlapping clones for expression from mammalian vectors.

FIG. 8: Nuclear fluorescence of COS-7 cells transformed with GFP-Drop1a including the two calponin homology domains at the aminoterminus of Drop1

FIG. 9: Monoclonal antibody mAB2.9, raised to the recombinant carboxyterminal spectrin-like domain of Drop1, recognizes this recombinant domain on immunoblots (left) On blots of SKOV-3 ovarian carcinoma cell lysates (center) it stains a high molecular band and various fragments in the pellet (P), and a high molecular weight band and various fragments in the pellet (P), and a large protein in the supernatant (S). In cytospins of the same cells it stains nuclei (right).

FIG. 10: Predicted amino acid sequence of Drop1 and predicted protein domains The ORF encodes a protein of 3032 amino acid residues. Analysis using SMART (Simple Modular Architecture Research Tool) software indicates two calponin homology (CH) domains, an AAA, four coiled coil (CC) motifs and twelve spectrin-like (SPEC) regions, as highlighted in gray in the sequence and indicated in the margin.

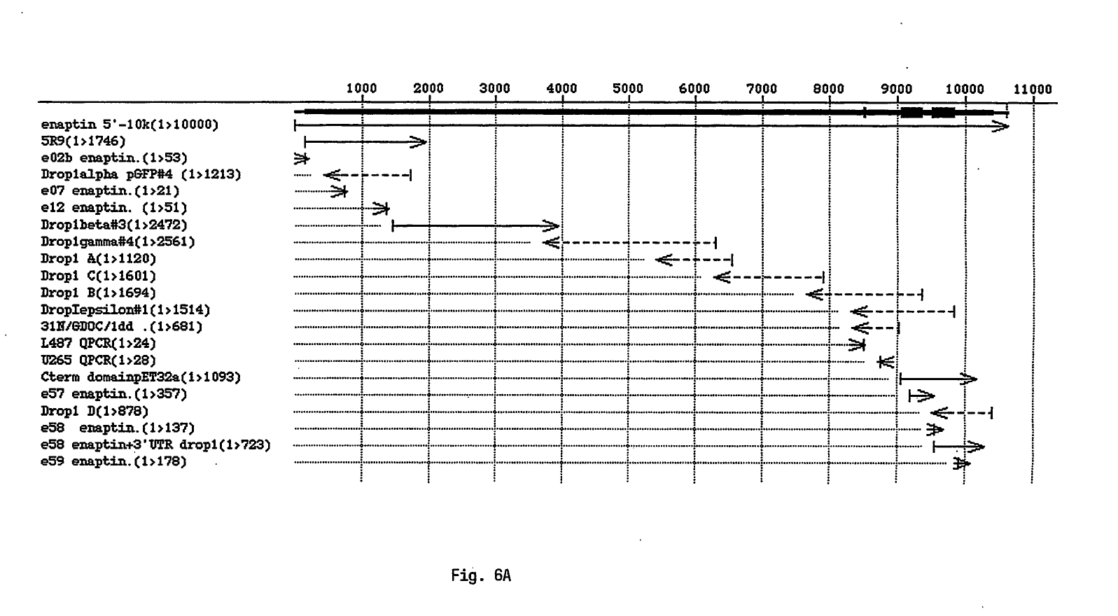

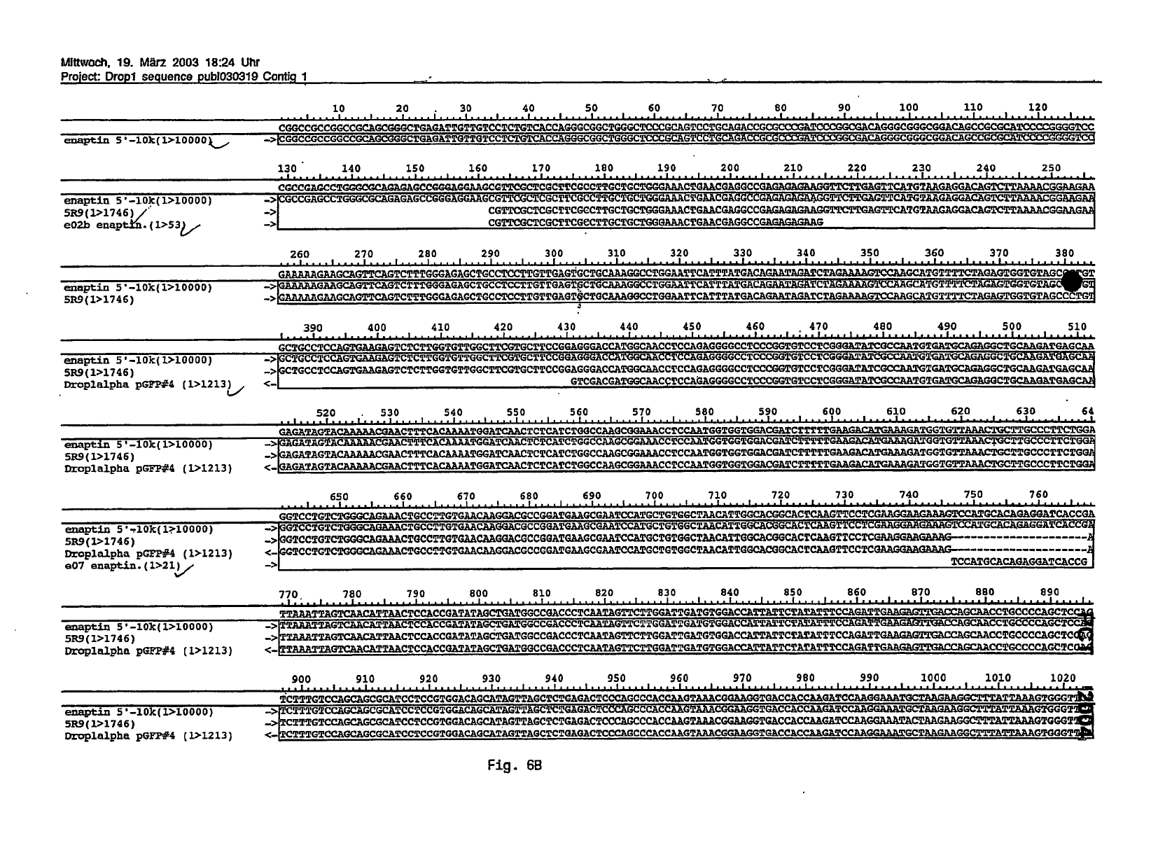

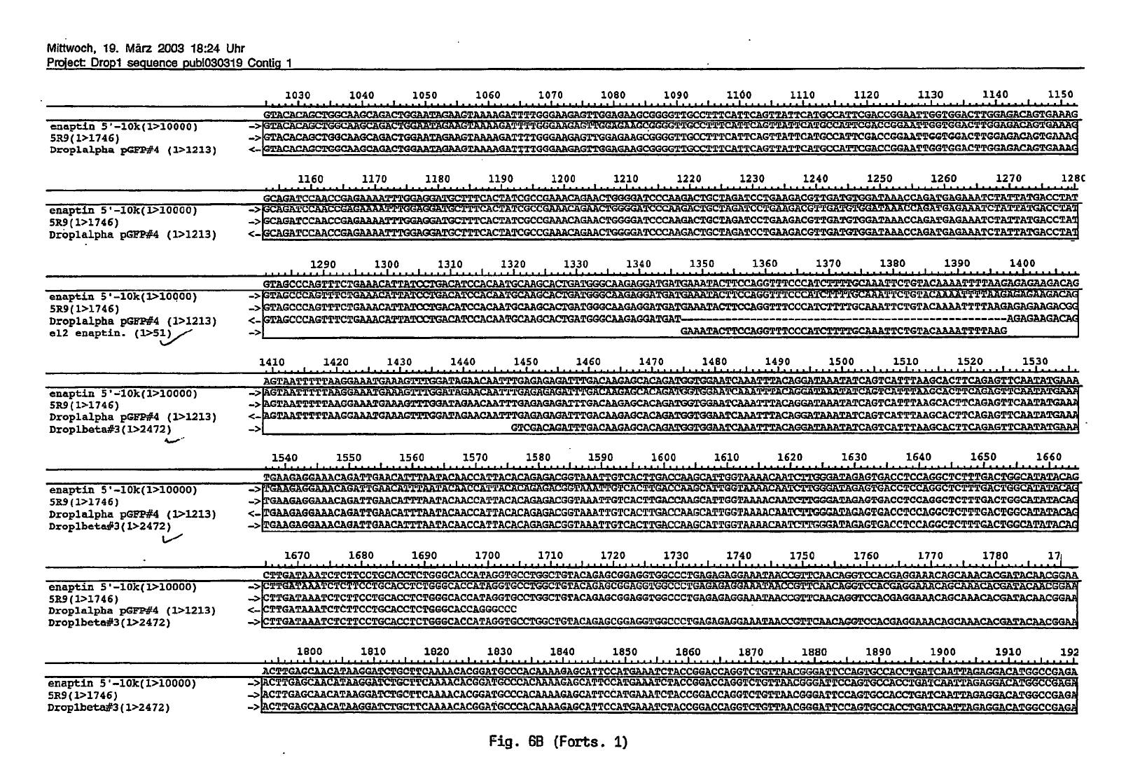

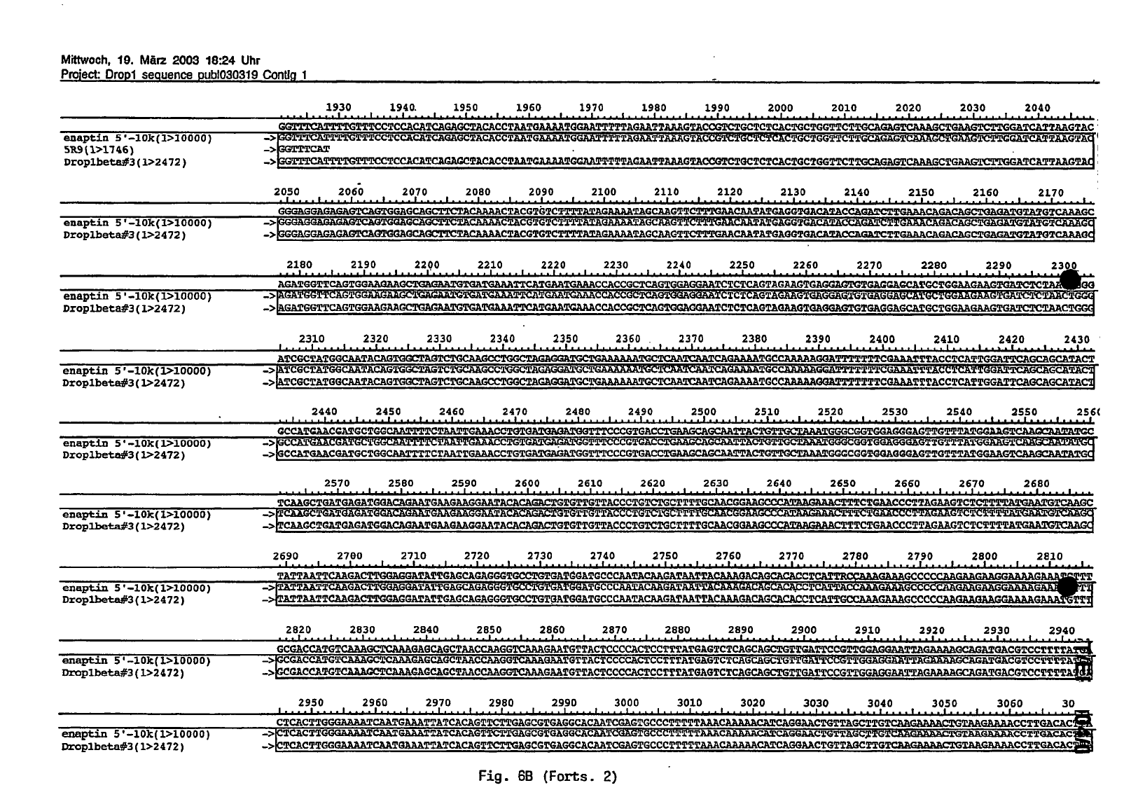

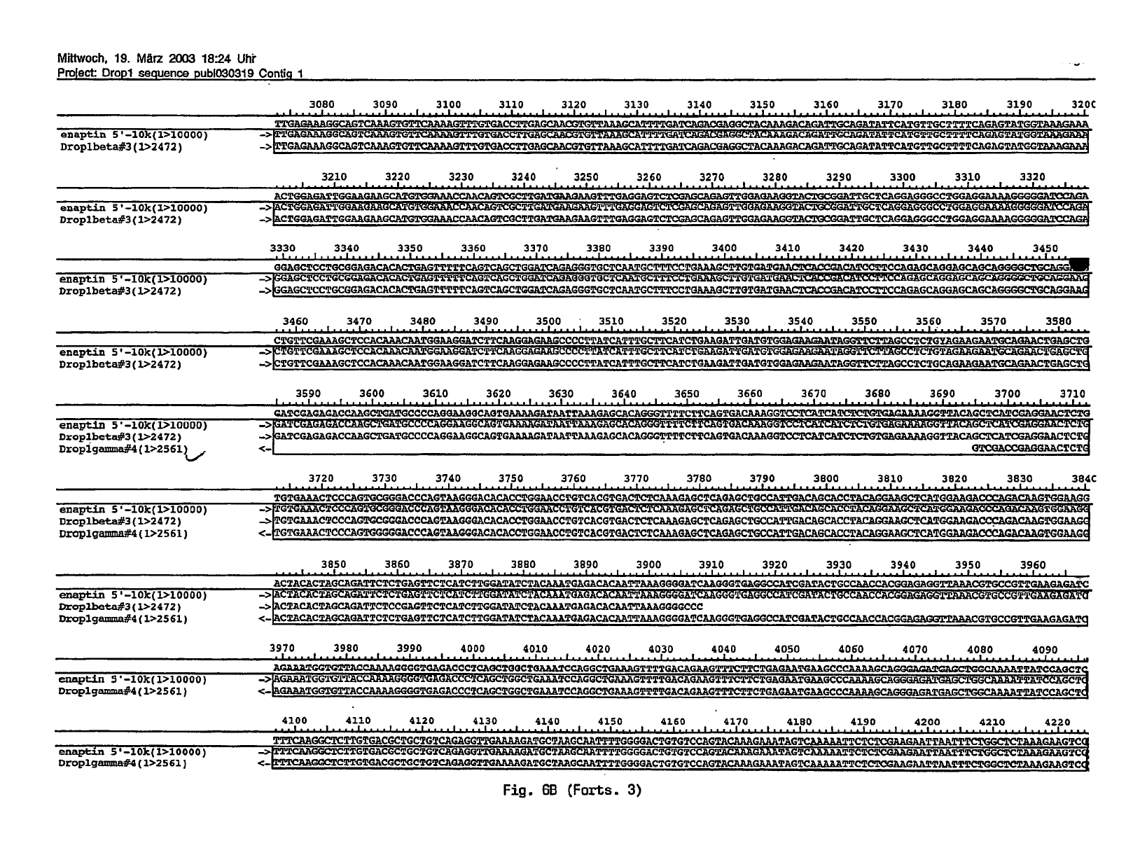

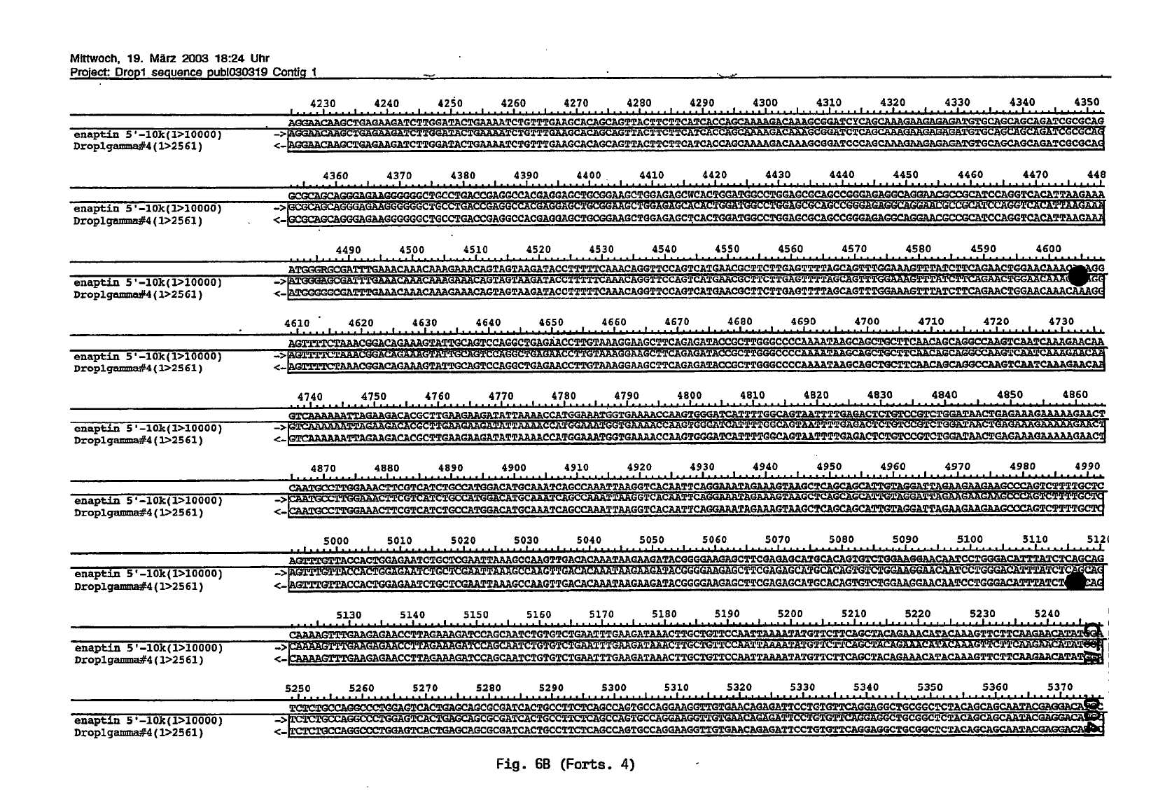

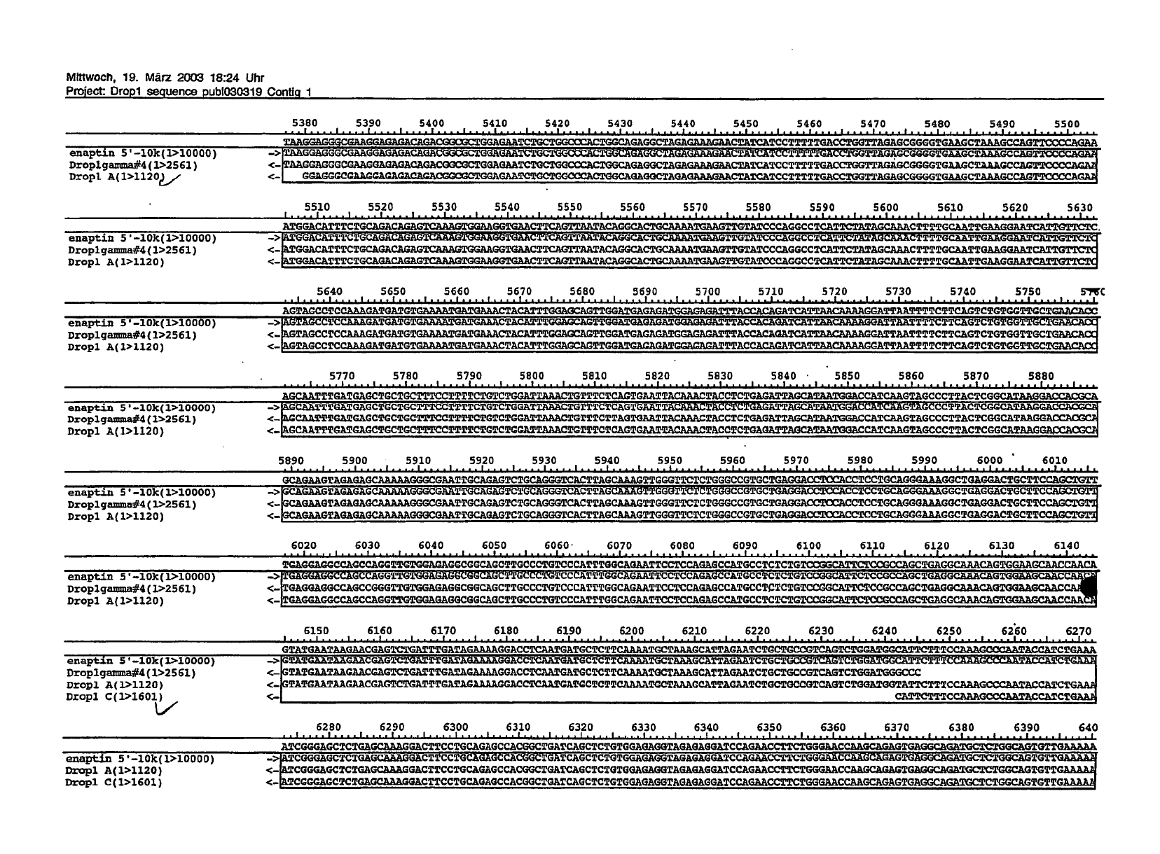

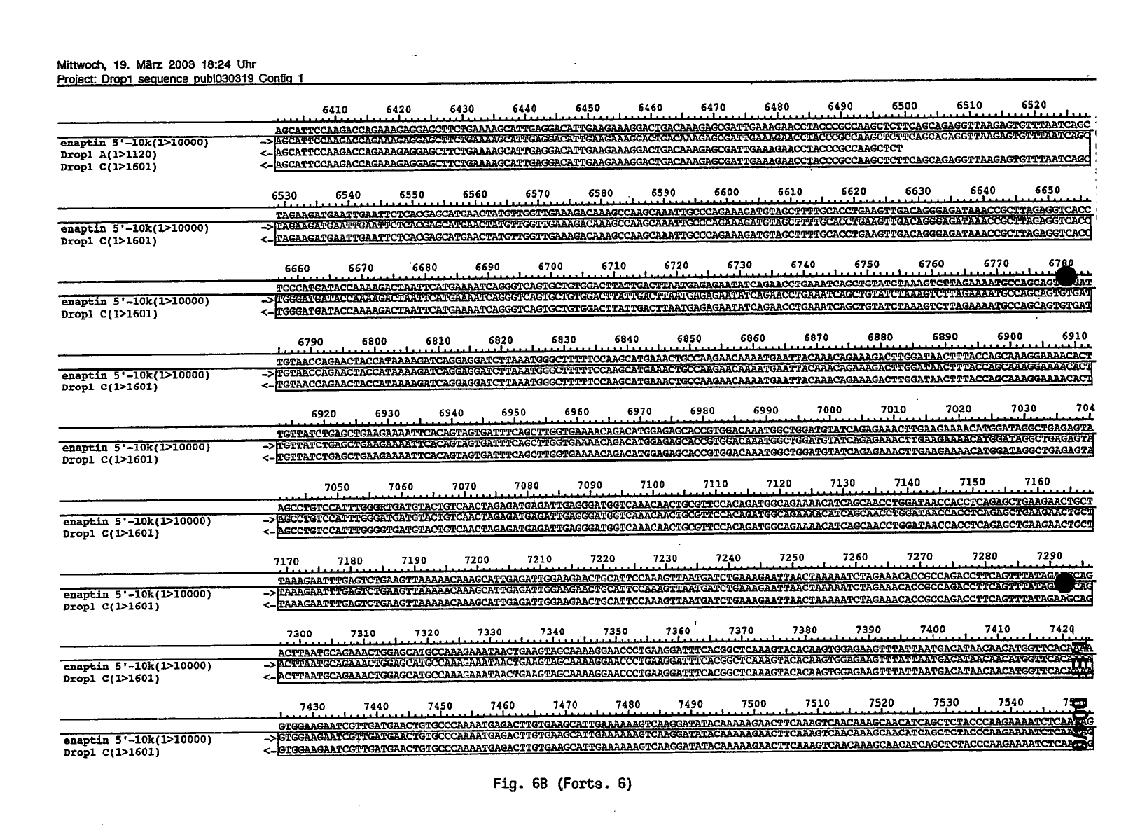

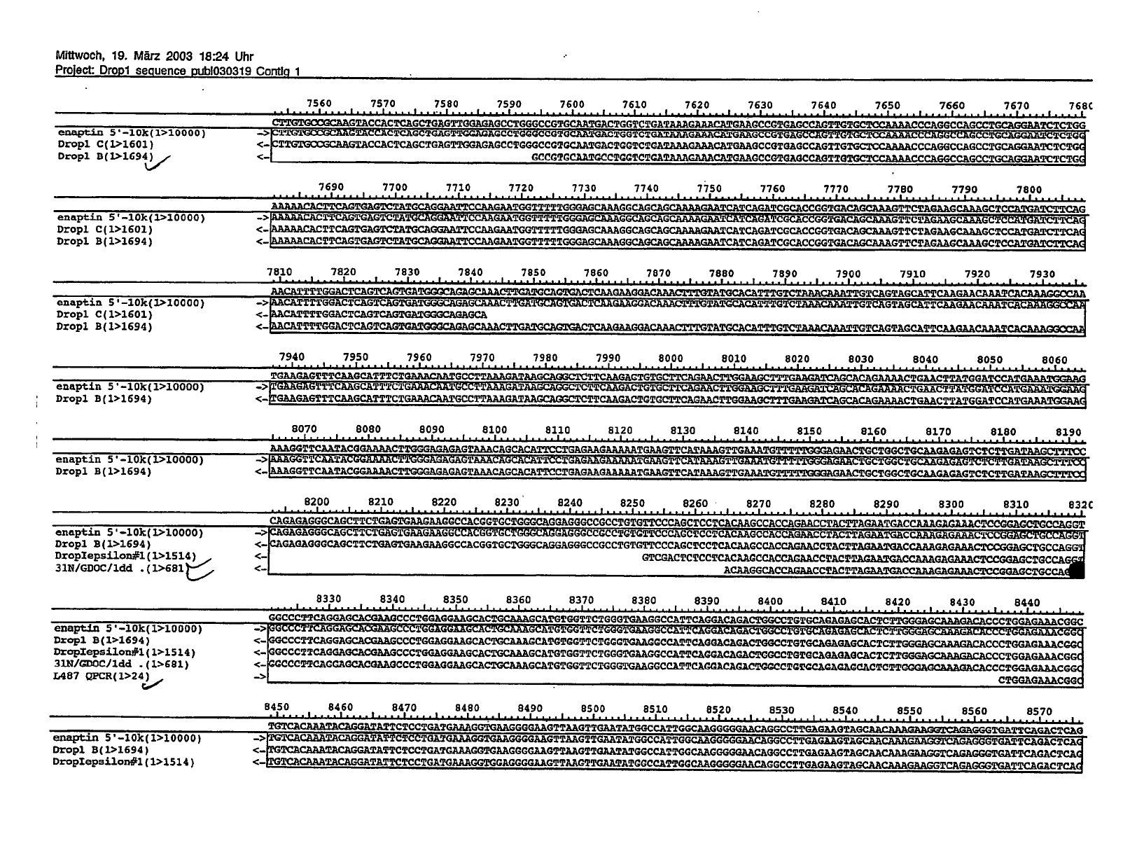

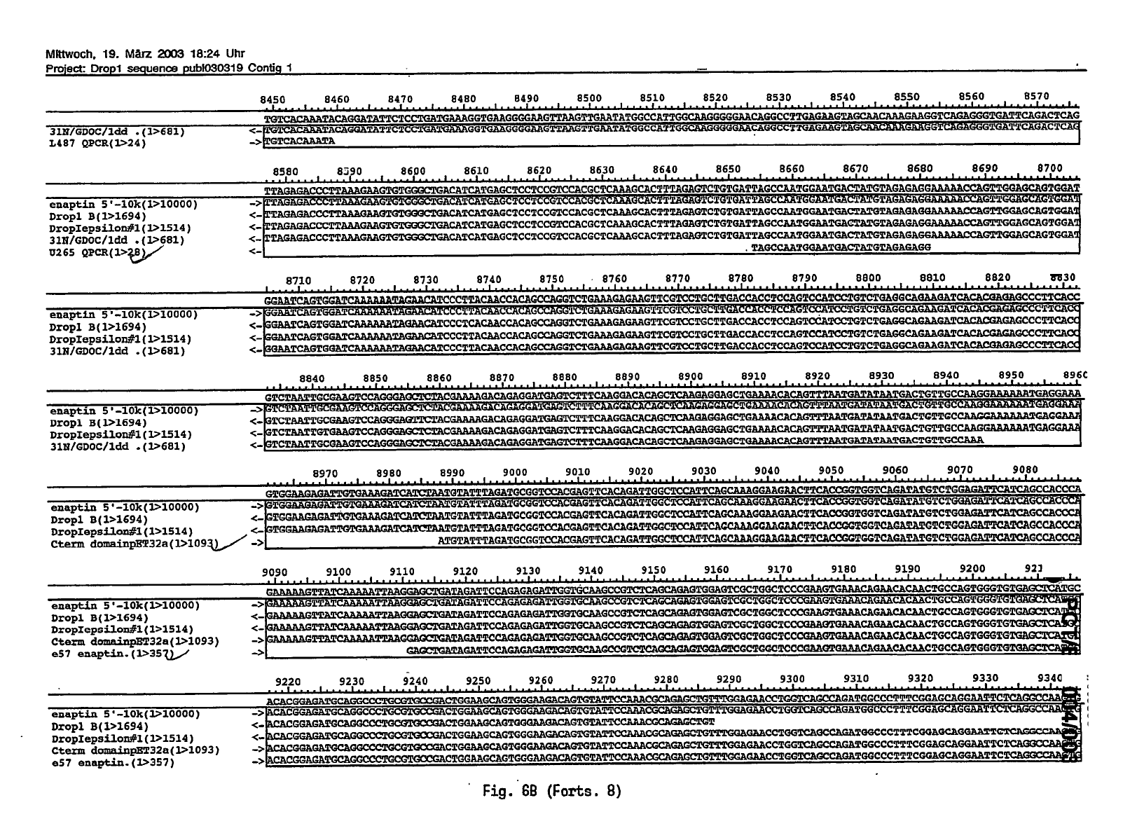

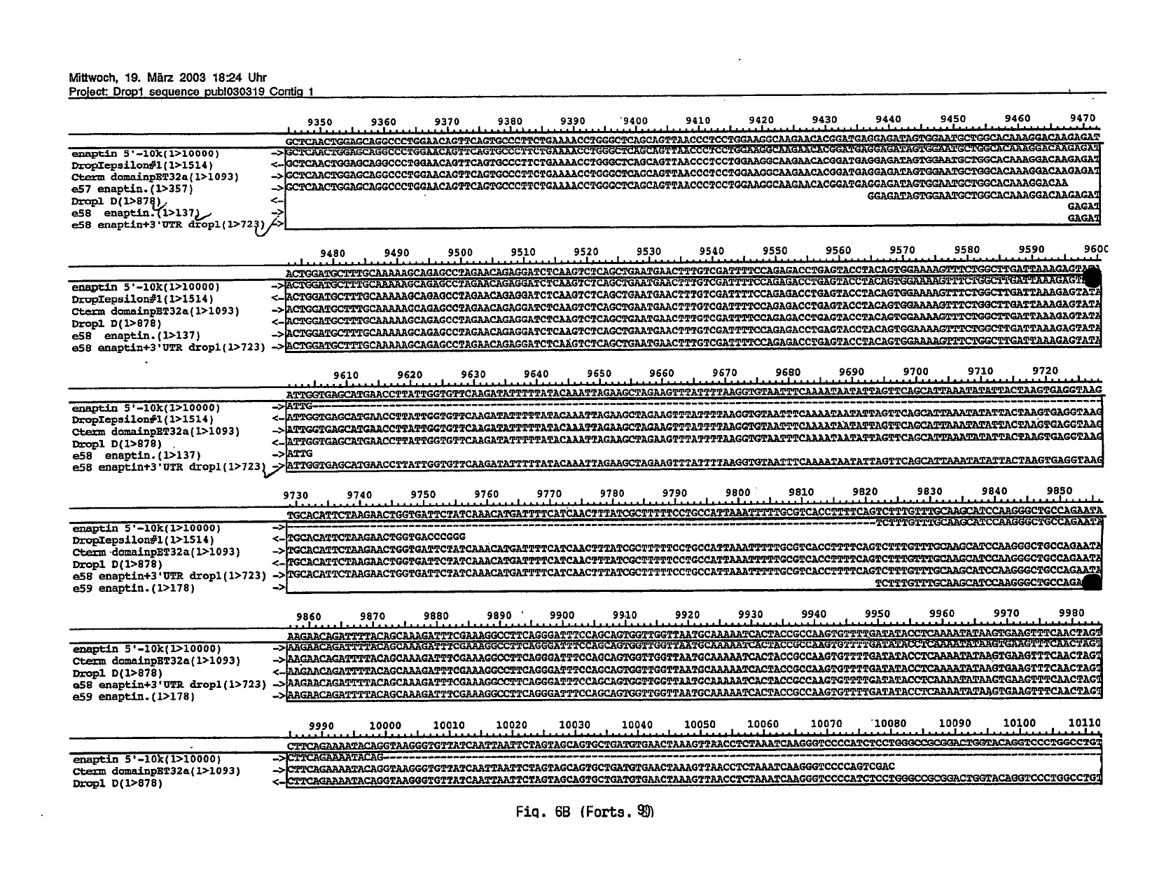

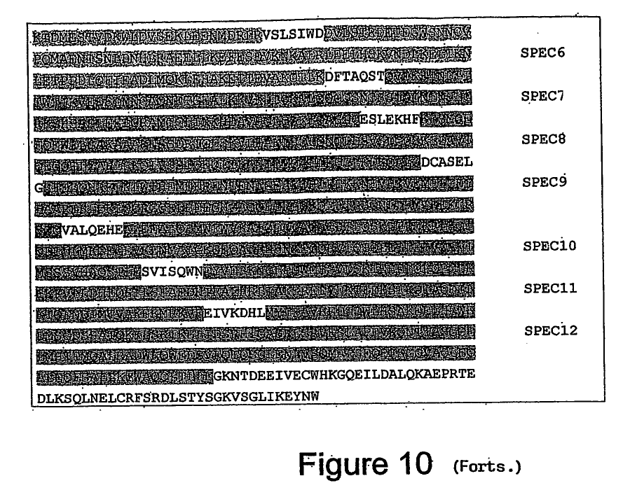

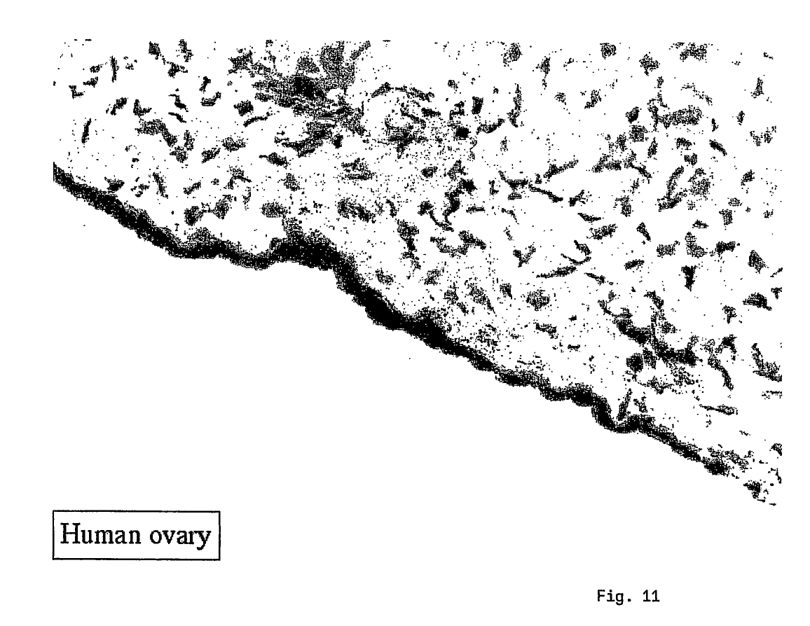

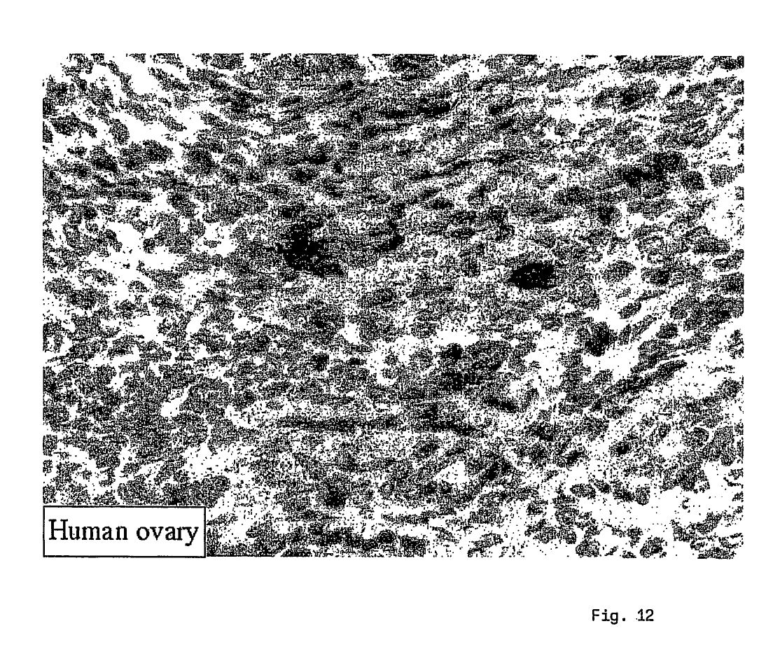

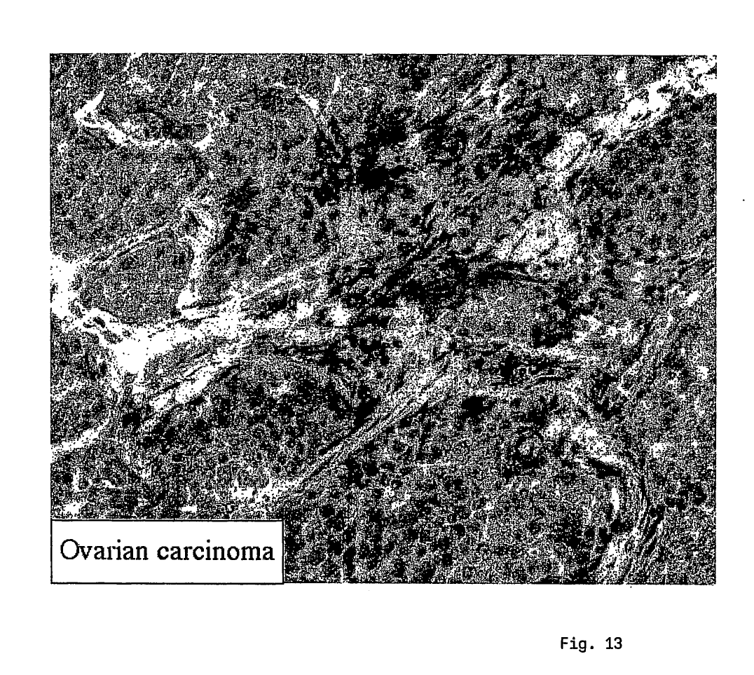

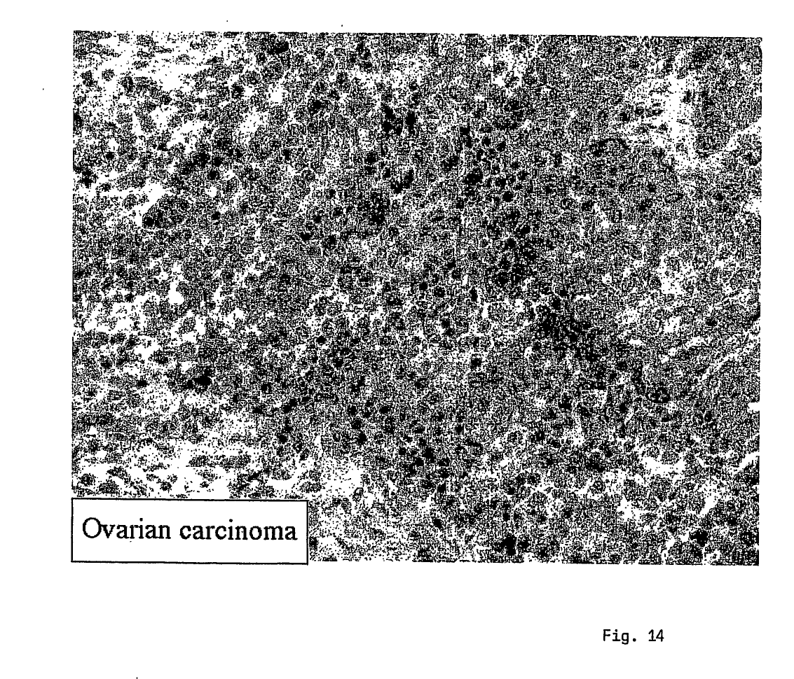

The present invention relates to a nucleic acid molecule encoding the human dystrophin-related polypeptide Drop1 or a polypeptide exhibiting a biological property of Drop1, which is:
(a) a nucleic acid molecule encoding a Drop1 polypeptide that consists of the amino acid sequence as depicted in FIG. 10;
(b) a nucleic acid molecule consisting of the nucleotide sequence as depicted in FIG. 6B;
(c) a nucleic acid molecule the sequence of which differs from the sequence of a nucleic acid molecule of (a) or (b) due to the degeneracy of the genetic code;
(d) a nucleic acid molecule, which represents a fragment of a nucleic acid molecule specified in (a) to (c); or
(e) a nucleic acid molecule complementary to the nucleic acid molecule specified in (a) to (d).

As used herein, a polypeptide exhibiting a biological property of Drop1 is understood to be a polypeptide having at least one of the activities of Drop1 as illustrated in the Examples, below, i.e. at least one biological activity of an ATPase.

In a first embodiment, the invention provides a nucleic acid molecule encoding a Drop1 polypeptide that consists of the amino acid sequence as depicted in FIG. 10.

The nucleic acid molecules of the invention can be both DNA and RNA molecules. Suitable DNA molecules are, for example, genomic or cDNA molecules. It is understood that all nucleic acid molecules encoding all or a portion of Drop1 are also included, as long as they encode a polypeptide with biological activity. The nucleic acid molecules of the invention can be isolated from natural sources or can be synthesised according to known methods.

The nucleic acid molecules of the present invention also include molecules which differ from the nucleic acid molecules with sequences shown in FIG. 6B due to the degeneracy of the genetic code.

In a further embodiment, the present invention provides nucleic acid molecules which comprise fragments of the nucleic acid molecules described above encoding a polypeptide of the invention. “Fragments” are understood to be parts of the nucleic acid molecules that are long enough to encode one of the described polypeptids. These fragments also comprise nucleic acid molecules specifically hybridizing to transcripts of the nucleic acid molecules of the invention. These nucleic acid molecules can be used, for example, as probes or primers in the diagnostic assay and/or kit described below and, preferably, are oligonucleotides having a length of at least 10, in particular of at least 15 and particularly preferred of at least 25 contiguous nucleotides of the nucleic acid molecule of the invention. The nucleic acid molecules and oligonucleotides (fragments) of the invention can also be used, for example, as primers for a PCR reaction.

In a still further embodiment, the present invention provides nucleic acid molecules which are complementary to the nucleic acid molecules of the invention.

Generally, by means of conventional molecular biological processes it is possible (see, e.g., Sambrook et al., supra) to introduce different mutations into the nucleic acid molecules of the invention. As a result Drop1 polypeptides or Drop1 related polypeptids with possibly modified biological properties are synthesised. One possibility is the production of deletion mutants in which nucleic acid molecules are produced by continuous deletions from the 5′- or 3′-terminal of the coding DNA sequence and that lead to the synthesis of polypeptids that are shortened accordingly. Another possibility is the introduction of single-point mutation at positions where a modification of the amino aid sequence influences, e.g., the proteolytic properties. By this method muteins can be produced, for example, that possess a modified Kₘ-value or that are no longer subject to the regulation mechanisms that normally exist in the cell, e.g. with regard to allosteric regulation or covalent modification.

For the manipulation in prokaryotic cells by means of genetic engineering the nucleic acid molecules of the invention or parts of these molecules can be introduced into plasmids allowing a mutagenesis or a modification of a sequence by recombination of DNA sequences. By means of conventional methods (cf. Sambrook et al., supra) bases can be exchanged and natural or synthetic sequences can be added. In order to link the DNA fragments with each other adapters or linkers can be added to the fragments. Furthermore, manipulations can be performed that provide suitable cleavage sites or that remove superfluous DNA or cleavage sites. If insertions, deletions or substitutions are possible, in vitro mutagenesis, primer repair, restriction or ligation can be performed. As analysis method usually sequence analysis, restriction analysis and other biochemical or molecular biological methods are used.

The polypeptids encoded by the various variants of the nucleic acid molecules of the invention show certain common characteristics, such as ATPase activity, molecular weight, immunological reactivity or conformation or physical properties like the electrophoretical mobility, chromatographic behavior, sedimentation coefficients, solubility, spectroscopic properties, stability, pH optimum, temperature optimum.

The invention furthermore relates to recombinant vectors containing the nucleic acid molecules of the invention. Preferably, they are plasmids, cosmids, viruses, bacteriophages and other vectors usually used in the field of genetic engineering. Vectors suitable for use in the present invention include, but are not limited to the T7-based expression vector for expression in mammalian cells and baculovirus-derived vectors for expression in insect cells. Preferably, the nucleic acid molecule of the invention is operatively linked to the regulatory elements in the recombinant vector of the invention that guarantee the transcription and synthesis of an mRNA in prokaryotic and/or eukaryotic cells that can be translated. The nucleotide sequence to be transcribed can be operably linked to a promoter like a T7, metallothionein I or polyhedrin promoter.

In a further embodiment, the present invention relates to recombinant host cells transiently or stably containing the nucleic acid molecules or vectors of the invention. A host cell is understood to be an organism that is capable to take up in vitro recombinant DNA and, if the case may be, to synthesize the polypeptids encoded by the nucleic acid molecules of the invention. Preferably, these cells are prokaryotic or eukaryotic cells, for example mammalian cells, bacterial cells, insect cells or yeast cells. The host cells of the invention are preferably characterized by the fact that the introduced nucleic acid molecule of the invention either is heterologous with regard to the transformed cell, i.e. that it does not naturally occur in these cells, or is localized at a place in the genome different from that of the corresponding naturally occurring sequence.

A further embodiment of the invention relates to a polypeptide exhibiting a biological property of the human dystrophin-related polypeptide Drop1 and being encoded by the nucleic acid molecules of the invention, as well as to methods for their production, whereby, e.g., a host cell of the invention is cultivated under conditions allowing the synthesis of the polypeptide and the polypeptide is subsequently isolated from the cultivated cells and/or the culture medium. Isolation and purification of the recombinantly produced polypeptide may be carried out by conventional means including preparative chromatography and affinity and immunological separations using, e.g., an anti-Drop1 antibody, or, e.g., can be substantially purified by the one-step method described in Smith and Johnson, Gene 67; 31-40 (1988). These polypeptides, however, not only comprise recombinantly produced polypeptides but include isolated naturally occurring polypeptides, synthetically produced polypeptides, or polypeptides produced by a combination of these methods. Means for preparing such polypeptides or related polypeptides are well understood in the art. These polypeptides are preferably in a substantially purified form.

The present invention also relates to an antibody that binds specifically to a Drop1 polypeptide or, a related polypeptide as defined above. Cells producing said antibody (DROP1-2.9) have been deposited on Mar. 26, 2003 according to the Budapest Treaty with the Deutsche Sammlung von Mikrorganismen und Zellkulturen, Mascheroder Weg, Braunschweig, Germany under DSM ACC2585. The term “antibody”, preferably, relates to antibodies which consist essentially of pooled monoclonal antibodies with different epitopic specifities, as well as distinct monoclonal antibody preparations. Monoclonal antibodies are made from an antigen containing fragments of the polypeptides of the invention by methods well known to those skilled in the art (see, e.g., Köhler et al., Nature 256 (1975), 495). As used herein, the term “antibody” (Ab) or “monoclonal antibody” (Mab) is meant to include intact molecules as well as antibody fragments (such as, for example, Fab and F(ab′) 2 fragments) which are capable of specifically binding to protein. Fab and f(ab′)2 fragments lack the Fc fragment of intact antibody, clear more rapidly from the circulation, and may have less non-specific tissue binding than an intact antibody. (Wahl et al., J. Nucl. Med. 24:316-325 (1983)). Thus, these fragments are preferred, as well as the products of a FAB or other immunoglobulin expression library. Moreover, antibodies of the present invention include chimerical, single chain, and humanized antibodies.

For certain purposes, e.g. diagnostic methods, the antibody of the present invention can be detectably labeled, for example, with a radioisotope, a bioluminescent compound, a chemiluminescent compound, a fluorescent compound, a metal chelate, or an enzyme.

The invention also relates to a transgenic non-human animal such as transgenic mouse, rats, hamsters, dogs, monkeys, rabbits, pigs, C. elegans and fish such as torpedo fish comprising a nucleic acid molecule or vector of the invention, preferably wherein said nucleic acid molecule or vector is stably integrated into the genome of said non-human animal, preferably such that the presence of said nucleic acid molecule or vector leads to the expression of a Drop1 polypeptide (or related polypeptide) of the invention. Said animal may have one or several copies of the same or different nucleic acid molecules encoding one or several forms of a Drop1 polypeptide or mutant forms thereof. This animal has numerous utilities, including as a research model for development/progression of carcinomas and therefore, presents a novel and valuable animal in the development of therapies, treatment, etc. for carcinomas associated with downregulation of Drop1, e.g., epithelial tumors. Accordingly, in this instance, the non-human mammal is preferably a laboratory animal such as a mouse or rat.

Preferably, the transgenic non-human animal of the invention further comprises at least one inactivated wild type allele of the corresponding Drop1 encoding gene. This embodiment allows for example the study of the interaction of various mutant forms of Drop1 polypeptides on the onset of the clinical symptoms of a carcinoma. All the applications that have been herein before discussed with regard to a transgenic animal also apply to animals carrying two, three or more transgenes. It might be also desirable to inactivate Drop1 protein expression or function at a certain stage of development and/or life of the transgenic animal. This can be achieved by using, for example, tissue specific, developmental and/or cell regulated and/or inducible promoters which drive the expression of, e.g., an antisense or ribozyme directed against the RNA transcript encoding the Drop1 encoding mRNA; see also supra. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of a mutant Drop1 protein may be controlled by such regulatory elements.

Furthermore, the invention also relates to a transgenic mammalian cell which contains (preferably stably integrated into its genome) a nucleic acid molecule according to the invention or part thereof, wherein the transcription and/or expression of the nucleic acid molecule or part thereof leads to reduction of the synthesis of a Drop1 protein. In a preferred embodiment, the reduction is achieved by an anti-sense, sense, ribozyme, co-suppression and/or dominant mutant effect. “Antisense” and “antisense nucleotides” means DNA or RNA constructs which block the expression of the naturally occurring gene product.

The provision of the nucleic acid molecule according to the invention opens up the possibility to produce transgenic non-human animals with a reduced level of a Drop1 protein as described above. Techniques how to achieve this are well known to the person skilled in the art. These include, for example, the expression of antisense-RNA, ribozymes, of molecules which combine antisense and ribozyme functions and/or of molecules which provide for a co-suppression effect. When using the antisense approach for reduction of the amount of Drop1 proteins in cells, the nucleic acid molecule encoding the antisense-RNA is preferably of homologous origin with respect to the animal species used for transformation. However, it is also possible to use nucleic acid molecules which display a high degree of homology to endogenously occurring nucleic acid molecules encoding a Drop1 protein. In this case the homology is preferably higher than 80%, particularly higher than 90% and still more preferably higher than 95%. The reduction of the synthesis of a polypeptide according to the invention in the transgenic mammalian cells can lead to various physiological, developmental and/or morphological changes.

Thus, the present invention also relates to transgenic non-human animals comprising the above-described transgenic cells. These may show, for example, a deficiency in Drop1 due to the stable or transient presence of a foreign DNA resulting in at least one of the following features:
(a) disruption of (an) endogenous gene(s) encoding Drop1;
(b) expression of at least one antisense RNA and/or ribozyme against a transcript comprising a nucleic acid molecule of the invention;
(c) expression of a sense and/or non-translatable mRNA of the nucleic acid molecule of the invention;
(d) expression of an antibody of the invention;
(e) incorporation of a functional or non-functional copy of the regulatory sequence of the invention; or
(f) incorporation of a recombinant DNA molecule or vector of the invention.

Methods for the production of a transgenic non-human animal of the present invention, preferably transgenic mouse, are well known to the person skilled in the art. Such methods, e.g., comprise the introduction of a nucleic acid molecule or vector of the invention into a germ cell, an embryonic cell, stem cell or an egg or a cell derived therefrom. The non-human animal can be used in accordance with a screening method of the invention described herein and may be a non-transgenic healthy animal, or may have a disorder, preferably a disorder caused by at least one mutation in the Drop1 protein. Such transgenic animals are well suited for, e.g., pharmacological studies of drugs in connection with mutant forms of the above described Drop1 polypeptide. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe; see supra.

In a further aspect, the invention relates to a method of identifying activators/agonists of a Drop1 polypeptide comprising the steps of: (a) incubating a candidate compound with a polypeptide of the invention, b) assaying a biological activity, and (c) determining if a biological activity of the polypeptide of the invention has been altered.

Preferably, the screening for these compounds involves producing appropriate cells which express Drop1, either as a secreted protein or on the cell membrane. Preferred cells include cells from mammals, yeast, Drosophila, or E. coli. Cells expressing Drop1 (or cell membrane containing the expressed polypeptide) are then preferably contacted with a test compound potentially containing the molecule to observe binding, stimulation, or inhibition of activity of Drop1.

The assay may simply test binding of a candidate compound to Drop1, wherein binding is detected by a label, or in an assay involving competition with a labeled competitor. Further, the assay may test whether the candidate compound results in a signal generated by binding to Drop1.

Alternatively, the assay can be carried out using cell-free preparations, polypeptide/molecule affixed to a solid support, chemical libraries, or natural product mixtures. The assay may also simply comprise the steps of mixing a candidate compound with a solution containing Drop1, measuring Drop1/molecule activity or binding, and comparing the Drop1/molecule activity or binding to a standard.

Preferably, an ELISA assay can measure Drop1 level or activity in a sample (e.g., biological sample) using a monoclonal or polyclonal antibody. The antibody can measure Drop1 level or activity by either binding, directly or indirectly, to Drop1 or by competing with Drop1 for a substrate. All of these above assays can be used as diagnostic or prognostic markers. The molecules discovered using these assays can be used to treat disease or to bring about a particular result in a patient (e.g., elimination of an epithelial tumor or stop of progression of tumor growth) by activating the Drop1 molecule. Moreover, the assays can discover agents which may enhance the production of Drop1 from suitably manipulated cells or tissues.

The candidate compound may be a single compound or a plurality of compounds. The term “plurality of compounds” in a method of the invention is to be understood as a plurality of substances which may or may not be identical.

Said compound or plurality of compounds may be chemically synthesized or microbiologically produced and/or comprised in, for example, samples, e.g., cell extracts from, e.g., plants, animals or microorganisms. Furthermore, said compound(s) may be known in the art but hitherto not known to be capable of activating, Drop1 polypeptides. The reaction mixture may be a cell free extract or may comprise a cell or tissue culture. Suitable set ups for the method of the invention are known to the person skilled in the art and are, for example, generally described in Alberts et al., Molecular Biology of the Cell, third edition (1994) and in the appended examples. The plurality of compounds may be, e.g., added to the reaction mixture, culture medium, injected into a cell or otherwise applied to the transgenic animal. The cell or tissue that may be employed in the method of the invention preferably is a host cell, mammalian cell or non-human transgenic animal of the invention described in the embodiments hereinbefore.

If a sample containing a compound or a plurality of compounds is identified in the method of the invention, then it is either possible to isolate the compound from the original sample identified as containing the compound capable of activating Drop1, or one can further subdivide the original sample, for example, if it consists of a plurality of different compounds, so as to reduce the number of different substances per sample and repeat the method with the subdivisions of the original sample. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical.

Several methods are known to the person skilled in the art for producing and screening large libraries to identify compounds having specific affinity for a target. These methods include the phage-display method in which randomized peptides are displayed from phage and screened by affinity chromatography to an immobilized receptor; see, e.g., WO 91/17271, WO 92/01047, U.S. Pat. No. 5,223,409. In another approach, combinatorial libraries of polymers immobilized on a chip are synthesized using photolithography; see, e.g., U.S. Pat. No. 5,143,854, WO 90/15070 and WO 92/10092. The immobilized polymers are contacted with a labeled receptor and scanned for label to identify polymers binding to the receptor. The synthesis and screening of peptide libraries on continuous cellulose membrane supports that can be used for identifying binding ligands of the polypeptide of the invention and thus possible activators as described, for example, in Kramer, Methods Mol. Biol. 87 (1998), 25-39.

More recently, WO 98/25146 described further methods for screening libraries of complexes for compounds having a desired property, especially, the capacity to agonize, bind to, or antagonize a polypeptide or bits cellular receptor. The complexes in such libraries comprise a compound under test, a tag recording at least one step in synthesis of the compound, and a tether susceptible to modification by a reporter molecule. Modification of the tether is used to signify that a complex contains a compound having a desired property. The tag can be decoded to reveal at least one step in the synthesis of such a compound. Other methods for identifying compounds which interact with the polypeptides according to the invention or nucleic acid molecules encoding such molecules are, for example, the in vitro screening with the phage display system as well as filter binding assays or “real time” measuring of interaction using, for example, the BIAcore apparatus (Pharmacia).

All these methods can be used in accordance with the present invention to identify activators/agonists of the Drop1 polypeptide or related polypeptide of the invention.

The nucleic acid molecule of the invention can also serve as a target for activators. Activators may comprise, for example, proteins that bind to the MRNA of a gene encoding a Drop1 polypeptide of the invention, thereby stabilizing the native conformation of the mRNA and facilitating transcription and/or translation, e.g., in like manner as Tat protein acts on HIV-RNA. Furthermore, methods are described in the literature for identifying nucleic acid molecules such as an RNA fragment that mimics the structure of a defined or undefined target RNA molecule to which a compound binds inside of a cell resulting in retardation of cell growth or cell death; see, e.g., WO 98/18947 and references cited therein. These nucleic acid molecules can be used for identifying unknown compounds of pharmaceutical interest, and for identifying unknown RNA targets for use in treating a disease. These methods and compositions can be used in screening for identifying compounds useful to alter expression levels of proteins encoded by a nucleic acid molecule. Alternatively, for example, the conformational structure of the RNA fragment which mimics the binding site can be employed in rational drug design to modify known antibiotics to make them bind more avidly to the target. One such methodology is nuclear magnetic resonance (NMR), which is useful to identify drug and RNA conformational structures. Still other methods are, for example, the drug design methods as described in WO 95/35367 or U.S. Pat. No. 5,322,933, where the crystal structure of the RNA fragment can be deduced and computer programs are utilized to design novel binding compounds which can act as antibiotics.

The identification of nucleic acid molecules which encode polypeptides which interact with the Drop1 polypeptides described above can also be achieved, for example, as described in Scofield (Science 274 (1996), 2063-2065) by use of the so-called yeast “two-hybrid system”. In this system the polypeptide encoded by a nucleic acid molecule according to the invention or a smaller part thereof is linked to the DNA-binding domain of the GAL4 transcription factor. A yeast strain expressing this fusion polypeptide and comprising a lacZ reporter gene driven by an appropriate promoter, which is recognized by the GAL4 transcription factor, is transformed with a library of cDNAs which will express plant proteins or peptides thereof fused to an activation domain. Thus, if a peptide encoded by one of the cDNAs is able to interact with the fusion peptide comprising a peptide of a Drop1 polypeptide of the invention, the complex is able to direct expression of the reporter gene. In this way the nucleic acid molecules according to the invention and the encoded peptide can be used to identify peptides and proteins interacting with Drop1 protein.

Once the transacting factor is identified, modulation of its binding to or regulation of expression of the Drop1 polypeptide of the invention can be pursued, beginning with, for example, screening for inhibitors against the binding of the transacting factor to the protein of the present invention. Activation of Drop1 proteins could then be achieved in animals by applying the transacting factor or the gene encoding it, e.g. in an expression vector. Furthermore, upon identification of the transacting factor, further components in the pathway leading to activation (e.g. signal transduction) or repression of a gene involved in the control of Drop1 then can be identified. Modulation of the activities of these components can then be pursued, in order to develop additional drugs and methods for enhancing the activity/amount of Drop1 . Thus, the present invention also relates to the use of the two-hybrid system as defined above for the identification of activators of Drop1.

The compounds isolated by the above methods also serve as lead compounds for the development of analog compounds. The analogs should have a stabilized electronic configuration and molecular conformation that allows key functional groups to be presented to the Drop1 or its possible receptor in substantially the same way as the lead compound. In particular, the analog compounds have spatial electronic properties which are comparable to the binding region, but can be smaller molecules than the lead compound, frequently having a molecular weight below about 2 kD and preferably below about 1 kD. Identification of analog compounds can be performed through use of techniques such as self-consistent field (SCF) analysis, configuration interaction (CI) analysis, and normal mode dynamics analysis. Computer programs for implementing these techniques are available; e.g., Rein, Computer-Assisted Modeling of Receptor-Ligand Interactions (Alan Liss, New York, 1989). Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art; see also supra. Furthermore, peptidomimetics and/or computer aided design of appropriate derivatives and analogues can be used, for example, according to the methods described above.

Once the described compound has been identified and obtained, it is preferably provided in a therapeutically acceptable form.

Accordingly, the present invention also relates to a pharmaceutical composition comprising a nucleic acid molecule, polypeptide, recombinant vector, antibody, or activator/agonist according to the present invention and optionally a pharmaceutically acceptable excipient, diluent or carrier.

The nucleic acid molecule suitable for pharmaceutical or diagnostic purposes is defined to be
(a) a nucleic acid molecule encoding a Drop1 polypeptide that comprises the amino acid sequence as depicted in FIG. 10;
(b) a nucleic acid molecule comprising the nucleotide sequence as depicted in FIG. 6B;
(c) a nucleic acid molecule encoding a polypeptide the sequence of which shows at least 65% identify to the amino acid sequence of the polypeptide encoded by a nucleic acid molecule specified in (a) or (b);
(d) a nucleic acid molecule the sequence of which differs from the sequence of a nucleic acid molecule of (a) to (c) due to the degeneracy of the genetic code;
(e) a nucleic acid molecule, which represents a fragment of a nucleic acid molecule specified in (a) to (d); or
(f) a nucleic acid molecule complementary to the nucleic acid molecule specified in (a) to (e).

The present invention also provides the pharmaceutical or diagnostic use of nucleic acid molecules encoding a polypeptide the amino acid sequence of which shows an identity of at least 80%, in particular an identity of at least 90%, preferably of at least 95%, 96%, 97%, 98% or 99% to the amino acid sequence of the polypeptide of FIG. 10. The amino acid sequences of such polypeptides are characterized by deletion, substitution and/or insertion of amino acid residue(s) compared to the amino acid sequence shown in FIG. 10 or the result of recombination. They can be naturally occurring variations, for example sequences from other organisms, or mutations that cam either occur naturally or that have been introduced by genetic mutagenesis. In order to identify and isolate such nucleic acid molecules, the molecules of the invention or parts thereof can be used, for example by means of hybridization. As a hybridization probe nucleic acid molecules can be used, for example, that have exactly or basically the nucleic acid sequence as depicted in FIG. 6B, or parts of these sequences. The fragments that have been used as hybridization probes can be synthetic fragments that were produced by means of conventional synthetic methods and the sequence of which basically corresponds to the sequence of the FIG. 6B sequence.

As used herein, the term “hybridisation” has the meaning of hybridisation under conventional hybridisation conditions, preferably under stringent conditions as described, for example, in Sambrook et al., Molecular Cloning, A Laboratory Manual 2^{nd} edition (1989) Cold Spring Harbor Laboratory Press, Cold Spring Harbor, N.Y. However, in certain cases, a hybridizing nucleic acid molecule can also be detected at lower stringency hybridisation conditions. Changes in the stringency of hybridisation and signal detection are primarily accomplished through the manipulation of formamide concentration (lower percentages of formamide result in lowered stringency), salt conditions, or temperature. For example, lower stringency conditions include an overnight incubation at 37° C. in a solution comprising 6×SSPE (20×SSPE=3M NaCl; 9.2M NaH₂PO₄; 0.02M EDTA, pH7.4), 0.5% SDS, 30% formamide, 100 μg/ml salmon sperm blocking DNA, following by washes at 50° C. with 1×SSPE, 0.1% SDS. In addition, to achieve even lower stringency, washes performed following stringent hybridisation can be done at higher salt concentrations (e.g. 5×SSC). Variations in the above conditions may be accomplished through the inclusion and/or substitution of alternate blocking reagents used to suppress background in hybridisation experiments. The inclusion of specific blocking reagents may require modification of the hybridisation conditions described above, due to problems with compatibility.

Examples of suitable pharmaceutical carriers etc. are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Administration of the suitable compositions may be effected by different ways, e.g. by intravenous, intraperetoneal, subcutaneous, intramuscular, topical or intradermal administration. The route of administration, of course, depends on the nature of the tumor, its localisation and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of an epithelial tumor, general health and other drugs being administered concurrently.

The delivery of the nucleic acid molecules of the invention can be achieved by direct application or, preferably, by using a recombinant expression vector such as a chimeric virus containing these compounds or a colloidal dispersion system. Direct application to the target site can be performed, e.g., by ballistic delivery, as a colloidal dispersion system or by catheter to a site in artery. The colloidal dispersion systems which can be used for delivery of the above nucleic acid molecules include macromolecule complexes, nanocapsules, microspheres, beads and lipid-based systems including oil-in-water emulsions (mixed), micelles, liposomes and lipoplexes, The preferred colloidal system is a liposome. The composition of the liposome is usually a combination of phospholipids and steroids, especially cholesterol. The skilled person is in a position to select such liposomes which are suitable for the delivery of the desired nucleic acid molecule. Organ-specific or cell-specific liposomes can be used in order to achieve delivery only to the desired tumor. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example by coupling the liposome to a specific ligand, e.g., an antibody, a receptor, sugar, glycolipid, protein etc., by well known methods). In the present invention monoclonal antibodies are preferably used to target liposomes to specific epithelial tumors via specific cell-surface ligands.

Preferred recombinant vectors useful for gene therapy are viral vectors, e.g. adenovirus, herpes virus, vaccinia, or, more preferably, an RNA virus such as a retrovirus. Even more preferably, the retroviral vector is a derivative of a murine or avian retrovirus. Examples of such retroviral vectors which can be used in the present invention are: Moloney murine leukemia virus (MoMuLV), Harvey murine sarcoma virus (HaMuSV), murine mammary tumor virus (MuMTV) and Rous sarcoma virus (RSV). Most preferably, a non-human primate retroviral vector is employed, such as the gibbon ape leukemia virus (GaLV), providing a broader host range compared to murine vectors. Since recombinant retroviruses are defective, assistance is required in order to produce infectious particles. Such assistance can be provided, e.g., by using helper cell lines that contain plasmids encoding all of the structural genes of the retrovirus under the control of regulatory sequences within the LTR. Suitable helper cell lines are well known to those skilled in the art. Said vectors can additionally contain a gene encoding a selectable marker so that the transduced cells can be identified. Moreover, the retroviral vectors can be modified in such a way that they become target specific. This can be achieved, e.g., by inserting a polynucleotide encoding a sugar, a glycolipid, or a protein, preferably an antibody. Those skilled in the art know additional methods for generating target specific vectors. Further suitable vectors and methods for in vitro- or in vivo-gene therapy are described in the literature and are known to the persons skilled in the art; see, e.g., WO 97/00957.

In order to achieve expression only in the target organ, e.g., an epithelial tumor to be treated, the nucleic acid molecules of the present invention can be linked to a tissue specific promoter and used for gene therapy. Such promoters are well known to those skilled in the art (see e.g. Zimmermann et al., (1994) Neuron 12, 11-24; Vidal et al.; (1990) EMBO J. 9, 833-840).

The present invention also relates to the use of the above compounds of the invention for the preparation of a pharmaceutical composition for treatment of an epithelial tumor, preferably for the treatment of ovarian, mammary, stomach, kidney, thyroid, pancreas, cervical, testis or lung carcinomas.

Recombinant re-expression of a protein which got lost or lost its activity by mutations during tumor progression is a strategy which has been clinically evaluated for ovarian carcinoma. Wild-type P53 was re-introduced into mutant cells in a phase I clinical trial using a viral vector. This gave a proof of principle that gene therapy is a promising strategy in ovarian cancer. The predominantly intraperitoneal pattern of metastasising of ovarian carcinoma makes it prone for intraperitoneal application of the therapeutic agent which increases the safety of the therapy and should lead to only moderate toxicity of such therapies. Considering the size of Drop1, even transfection of defective cells with cDNA fragments may have a therapeutic effect. This strategy has already been used successfully with muscle dystrophies in animal models (Chamberlain, Hum. Mol. Genet. 11 (2002), 2355-2362; Scott et al., Neuromuscular Disord. 12 (2002), S23-S29). In addition, it can be expected that expression of Drop1 can be upregulated in tumors by therapeutic use of demethylating drugs.

Finally, the present invention also relates to a method of diagnosing an epithelial tumor or a susceptibility to an epithelial tumor in a subject comprising:
(a) determining the presence or amount of expression of the Drop1 polypeptide in a biological sample; and
(b) diagnosing an epithelial tumor or a susceptibility to an epithelial tumor based on the presence or amount of expression of the Drop1 polypeptide.

The present invention also relates to a diagnostic kit useful for the detection and/or characterization of an epithelial tumor or a predisposition to such a tumor, e.g., an ovarian or mammary carcinoma, containing a nucleic acid molecule, polypeptide and/or antibody of the invention. Such kits are useful for the detection of a target cellular component, which is Drop1 or, alternatively, Drop1 encoding mRNA, wherein an decreased concentration of Drop1 (compared to the concentration in normal tissue) or, alternatively, Drop1 encoding mRNA is indicative for a epithelial tumor or a disposition to such a tumor.

The Drop1 polypeptide or the corresponding mRNA, e.g. in biological fluids or tissues, may be detected directly in situ, e.g. by in situ hybridisation (e.g., according to the examples, below) or it may be isolated from other cell components by common methods known to those skilled in the art before contacting with a probe. Detection methods include Northern Blot analysis, RNase protection, in situ methods, e.g. in situ hybridisation, in vitro amplification methods (PCR, LCR, QRNA replicase or RNA-transcription/amplification (TAS, 3SR), reverse dot blot disclosed in EP-B1 0 237 362)), immunoassays, Western Blot and other detection assays that are known to those skilled in the art. Preferably, these detection methods are in an array formate.

The probe (e.g. a specific antibody or specific oligonucleotide) of the diagnostic kit can be detectably labeled. In a preferred embodiment, said kit contains an anti-Drop1 antibody and allows said diagnosis, e.g., by ELISA and contains the antibody bound to a solid support, for example, a polystyrene microtiter dish or nitrocellulose paper, using techniques known in the art. Alternatively, said kits are based on a RIA and contain said antibody marked with a radioactive isotope. In a preferred embodiment of the kit of the invention the antibody is labeled. Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium rhodamine, and biotin. In addition to assaying Drop1 levels in a biological sample, the polypeptide can also be detected in vivo by imaging. Antibody labels or markers for in vivo imaging of protein include those detectable by X-radiography, NMR or ESR. For X-radiography, suitable labels include radioisotopes such as barium or caesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma. A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments”. (Chapter 13 in Tumor Imaging: The Radiochemical Detection of Cancer, S. W. Burchiel and B. A. Rhodes, eds., Masson Publishing Inc. (1982)).

For diagnostic purposes, preferably the probes (e.g., antibodies or oligonucleotides) of the invention do not bind to enaptin and do not hybridize with enaptin encoding mRNA, respectively.

The marker Drop1 is also useful for prognosis, for monitoring the progression of an epithelial tumor, for monitoring the therapeutic success during and/or after a tumor therapy, for predictive purposes and the diagnostic evaluation of the degree of malignancy of the tumor (grading and staging), e.g. by using in situ hybridisation, e.g. according to the examples below.

In addition, the inventors have discovered that if the Drop 1 promotor is hypermethylated, Drop 1 will not be expressed or it will be expressed on a lower level (i.e. it is downregulated). Thus, a therapeutic approach is the use of methyltransferase inhibitors. Furthermore, the promotor may be used for epigenomic diagnostic, preferably on an array formate.

The following examples illustrate the invention.

### EXAMPLE 1

### General Methods

(A) Reaction Kits

ABI PRISM-BigDye-Terminator-Cycle-Sequencing-Ready-Reaction-kit, Version 2 (Applied Biosystems, Weiterstadt, Germany); DIG RNA Labeling Kit (Roche Diagnostics, Mannheim, Germany); DNA Lab Chip Kit (Agilent Technologies, Waldbronn, Germany); PCR Product Purification Kit (Roche Diagnostics); QIAprep Spin Miniprep Kit (Qiagen, Hilden, Germany); QIAquick Gel Extraction Kit (Qiagen); QIA shredder columns (Qiagen); Rneasy Mini Kit (Qiagen); TOPO TA Cloning Kit (Invitrogen, Karlsruhe, Germany); Qiagen PCR Cloning plus kit (Qiagen).

(B) Enzymes, Buffers, Reagents and Markers

Advantage-cDNA-Polymerase-Mix (Clontech, Heidelberg, Germany); anti-DIG-FAB-Fragments (Boehringer, Mannheim, Germany); BamHI-restriction enzyme and reactionbuffer (Gibco BRL, Eggenstein, Germany); cDNA PCR-Reaktionsbuffer (Clontech); Desoxyribonuklease I (Gibco BRL); DNTP Mix (Amersham, Cleveland); DTT (Sigma-Aldrich, Deisenhofen, Germany); Dulbeccois Modified Eagle Medium (DMEM) (Invitrogen, Karlsruhe, Germany); EcoRV-restriction enzyme (Gibco BRL); Fetal calf serum (FCS) (Bio Whittaker, Verviers); First strand buffer (Gibco BRL); DNA size standard: Gene Ruler 100 bp (MBI Fermentas, St. Leon Roth, Germany); Gene Ruler 1 kb (MBI Fermentas); Goldstar DNA-Polymerase (Eurogentec, Karlsruhe, Germany); Goldstar DNA-Polymerase-Buffer (Eurogentec); Phosphate Buffered Saline (PBS) (Invitrogen); Proteinase K (Sigma-Aldrich); RLT Lysis Buffer (Qiagen); RNAse A (Sigma-Aldrich); RNA size standard (Gibco BRL); Rnase inhibitor (Boehringer, Mannheim); RNAlater (ams, Wiesbaden, Germany); SuperSCRIPT II RNase H-Reverse Transcriptase (Gibco BRL); t-RNA (Sigma-Aldrich); Trypan blue (Biochrom, Berlin, Germany); Trypsin (Panbiotech, Aidenbach, Germany).

(C) Carcinoma Cell Lines

Cell lines CaOv-3, ES-2, MDAH 2774, NIH:OVCAR-3, SK-OV-3 were obtained from the American Type Culture Collection (Rockville, USA), GG HeST and MT were provided by Dr. M. Lindner, Oncology Laboratory of the Women's University Hospital in Heidelberg and Dr. B. Gückel, Women's University Hospital Tübingen

| Cell line | Characteristics |
|---|---|
| CaOv-3 | Derived from a human adenocarcinoma of the ovary |
| | grown as monolayer, |
| | epithelial-like morphology |
| ES-2 | Derived from a poorly differentiated human ovari [symbol] |
| | clear cell carcinoma, hyperdiploid karyotype |
| MDAH 2774 | human adenocarcinoma of the ovary, grown [symbol] |
| | monolayer with epithelial morphology |
| NIH: OVCAR-3 | Derived from the malignant ascites of a patie [symbol] |
| | with progressive adenocarcinoma of the ovary, gro [symbol] |
| | as monolayer with epithelial morphology, exhibi [symbol] |
| | androgen- and estrogen receptors, near triplo [symbol] |
| | karyotype, large number of structurally abnorm [symbol] |
| | and unassignable chromosomes. |
| SK-OV-3 | Derived from from malignant ascites of a patie [symbol] |
| | with adenocarcinoma of the ovary, grown [symbol] |
| | monolayer; epithelial-like morphology |
| GG | Derived from the malignant ascites of a patie [symbol] |
| | with poorly differentiated serous-papilla [symbol] |
| | cystadenocarcinoma of the ovary, stage FIGO II |
| | GIII, with metastases in the ovary, both fallopi [symbol] |
| | tubes, isthmus of the cervix uteri, perimetrium |
| | the corpus utery, bladder peritoneum and oment [symbol] |
| | majus. Grown as monolayer. |
| HeSt | Derived from the malignant ascites of a patie [symbol] |
| | with serous-papillary cystadenocarcinoma of t [symbol] |
| | ovary, stage FIGO III, grown as monolayer. |
| MT | Derived from the malignant ascites of a patie [symbol] |
| | with serous-papillary cystadenocarcinoma of t [symbol] |
| | ovary, stage FIGO III, grown as monolayer. |

(D) Procurement of Tissues

Upon agreement of the patient in writing, procurement of tumor and normal tissue was performed intraoperatively under sterile conditions at the Women's University Hospitals in Heidelberg and Tübingen. Epithelia were removed by 10 to 15 smears using a plastic loop. In cases were no healthy ovarian epithelium could be obtained, epithelium from the infundibulum tubae uterinae (the uppermost part of the fallopian tube) was used as a reference tissue. In terms of expression patterns this is the tissue most closely related to the ovarian surface epithelium. Each smear was immediately transferred to a 1.5 ml Eppendorf tube containing 350 μl of RNA-Later. These samples were stored at −80° C.

In addition, tissue blocks of solid carcinoma of tumor free ovarian tissue and of fallopian tube were procured by transferring them into Falcon tubes containing 4% paraformaldehyde buffer and were fixed for in-situ hybridisation. After fixation for 15 h at 4° C., the blocks were washed twice with PBS for 15 min each and stored in 70% ethanol at 4° C. Additional small blocks of tissue were procured for laser captured microdissection in cryotubes containing 350 μl of RNAlater, shock frozen in liquid nitrogen at −196° C. and stored at −80° C. Dehydrated frozen sections of 7 μm were microdissected using an Arcturus PixCell II microdissector.

(E) RNA Isolation, Quality Control, and Reverse Transcription

Samples in RNAlater were thawed. Three volumes of RLT lysis buffer containing 10% 2-mercaptoethanol were added, the cells were lysed by incubation for 30 min at 37° C. under gentle shaking and finally vortexed. Cell fragments were removed by passing the solution through a QIA shredder column for 2 min at 8,000 rpm in a microcentrifuge. The eluant was mixed with an equal volume of 70% ethanol and centrifuged through RNeasy mini spin columns for 15 sec at 8,000 rpm. The RNA bound to the columns was washed with 700 μl of RW1 buffer and twice with 500 μl RPE buffer containing ethanol. The RNeasy filters were dried by centrifugation for 2 min at maximum speed. RNA was eluted with 2×30 μl RNase free DEPC water at 8,000 rpm. RNA concentration was determined spectrophotometrically and adjusted to equal concentration for each individual pair of tissues. Its quality was assessed by agarose gel electrophoresis or by using an Agilent Bioanalyzer 2100. The ratio of 28S:18S rRNA should be close to 2:1. Residual DNA was digested with 2U of DNAse I for 15 min at 25° C. The reaction was terminated by addition of 1/10 vol. 25 mM EDTA pH 8.0 and denaturation for 10 min at 65° C. In addition, RNA was isolated from frozen tissue sections, microdissected by laser capture microdissection (Arcturus PixCell II). Integrity of the RNA was checked using the Agilent system. From this source, pA-RNA was reversely transcribed and used without amplification for Differential Display and quantitative PCR.

(F) Differential Display

Differential display was performed using a modification of Liang's and Pardee's method with a self-designed set of arbitrary primers. A strong difference in expression was found in patient 31 with the arbitrary decamer primer 5′ ACAAGGCACC 3′ ( designated DOC), and a decamer anchor primer 5′ TTTTTTTTCG 3′, resulting in a 680 bp PCR fragment in normal epithelium, which was absent from the pattern derived from the tumor of the same patient. This fragment was eluted from the gel, amplified, cloned into TOPORII, and sequenced.

(G) Validation of Differential Expression by Quantitative and Semi-Quantitative RT-PCR

The differential expression was validated by quantitative PCR and analyzed in the Agilent 2100 Bioanalyzer (Agilent Technologies, Waldbronn, Germany, PC Hewlett packard, Vectra VL, Hewlett packard, Boeblingen; Software Agilent 2100 Bio Sizing 1.3, Agilent Technologies, Waldbronn) or by semi-quantitative analysis of the resulting cDNA on a 2% agarose gel.

Conditions:

1 μl cDNA of the 20 μl RT-mixture (corresponding to 0.5 ng of cDNA);
1 μl Drop1 Upper primer (5′ CTGGAGAAACGGCTGTCACAA ATA 3′ corresponding to positions 8255-8278 in exon 53), 20 μM;
1 μl Drop1 Lower primer (Lower primer 5′ CCTCTCTACATAGTCATTCCATTGGCTA 3′, complementary to positions 8500-8473 in exon 55), 20 μM;
5 μl cDNA-PCR reaction buffer, 10×;
1 μl dNTP-Mix, 10 mM;
1 μl advantage-cDNA-polymerase mix, 50×;

40 μl aqua dest.

| advantage-cDNA polymerase mix | | | cDNA-PCR reaction buffer | | |
|---|---|---|---|---|---|
| Glycerol | 1.0% | | Tricine-KOH, pH 9.2, | 40 | mM |
| Tris-HCL, pH 7.5, | 0.8 | mM | KOAc | 15 | mM |
| KCL | 1.0 | mM | Mg(OAc)₂ | 3.5 | mM |
| (NH₄)₂SO₄ | 0.5 | mM | BSA | 3.75 | μg/ml |
| EDTA | 2.0 | μM | | | |
| β-Mercaptoethanol | 0.1 | mM | | | |

| | |
|---|---|
| Thesit | 0.005% |

The PCR reactions were performed in a PTC-200 Peltier Thermal Cycler (Biozym, Hess. Oldendorf, Germany) under the following conditions:

| | | |
|---|---|---|
| 94° C. | 4 min | Denaturation of DNA |
| 1. 94° C. | 1 min | Denaturation of DNA |
| 2. 58° C. | 40 sec | Annealing |
| 3. 72° C. | 1 min | Elongation |
| 4° C. | hold | |

Steps 1 to 3 were repeated 34 to 46 times. After individual cycles in that range, aliquots were removed and run on a gel. The amount of PCR-product from the first cycle producing a visible band of 246 bp was then quantitated using the Agilent system and an intercalating dye to detect double-stranded DNA. Under these conditions, quantitative PCR detected Drop1 plus enaptin, yet not cpg2, and the various nexins.

(H) Northern Blot Analysis and Cancer Profiling Array

Clone 3R9 had been generated in a 3′ RACE experiment using an ovarian RACE library. Two human multiple-tissue Northern blots (Clontech) were pre-hybridized in 0,5 M sodium phosphate, pH 7,2, 7% SDS, 1 mM EDTA and 0,1 mg/ml salmon sperm DNA at 65° C. for 1 h, and subsequently hybridized for 14 h at 65° C. in the same buffer containing the labelled probe at a concentration of 1,5×10⁶ cpm/ml. The filters were washed twice at 65° C. in a buffer containing 0,2×SSC and 0,2% SDS and exposed for 20 h. To confirm an equal loading of mRNA in each lane, the blots were hybridized with labelled human β-actin cDNA.

The cancer profiling array from BD Biosciences Clontech (Palo Alto) catalog # 7841-1 included normalized cDNA from 241 tumor and corresponding normal tissues from individual patients, amplified by SMART technology. Human ubiquitin cDNA control probe had been hybridized to the array to ensure equal loading.

(I) In Situ Hybridisation

In situ hybridisation was carried out as described by DeJong et al., 1985; Straus, 1982; Scopsi und Larson, 1986).

(J) Digoxigenin-Labeling of RNA

A digoxigenin-labeled RNA-probe was generated by in vitro-transcription of the 3R9 clone from PCRII-Topo (see above), into which the cDNA fragment had been cloned to produce cDNA for sequencing this product. The vector is suited for in vitro-transcription, as it has promotors for the phage RNA-polymerases SP6 und T7 on either side of the insert. Thus, after linearization at the restriction sites BamHI and EcoRV, two single-stranded RNA-probes of opposite orientation (“sense” and “antisense”) could be transcribed.

Linearization was assayed by gel electrophoresis of a small aliquot, and labeling was performed using the DIG-RNA-labeling-kit.

Digoxigenin-Labeling of RNA Using SP6-Polymerase after EcoRV Cleavage T7-Polymerase after BamHI-Cleavage

| | | | | | |
|---|---|---|---|---|---|
| linearized vector-DNA | 7.4 | μl (1 μg) | linearized vector-DNA | 5 | μl (1 μg) |
| transcription buffer, 10x | 2 | μl | transcription buffer, 10x | 2 | μl |
| Labeling-Mix, 10x | 2 | μl | Labeling-Mix, 10x | 2 | μl |
| RNase-Inhibitor, 20 U | 1 | μl | RNase-Inhibitor, 20 U | 1 | μl |
| SP6-polymerase, 40 U | 2 | μl | T7-polymerase, 40 U | 2 | μl |
| DEPC-H₂O | 5.6 | μl | DEPC-H₂O | 8 | μl |

The components were mixed on ice and incubated for 2 h at 37° C. To assess the quality and the amount of the resulting single-stranded RNA, 1 μl of the product was run on a 2% TAE-agarose gel. Then, 2 μl (=2U) RNase-free DNase I was added and incubated at 37° C. for 15 min, to destroy the vector and avoid disturbance of the hybridisation by unlabeled DNA. The reaction was stopped by adding 2 μl of 0,5 mM EDTA pH 8, and the probes were stored at −20° C.

(K) Preparation of Tissue Sections

Procured tissues that had been stored in 70% ethanol/PBS were washed in 10% Ethanol/PBS at 25° C. and dehydrated by subsequent washes in 85%, 95% ethanol/PBS, 3×100% ethanol at 25° C., 2× in xylol, and incubated in xylol for 15 h at 25° C. Subsequently the tissues were incubated for 60 min at 56° C. in a paraffin/xylol-mixture, and covered with pure paraffin for 3× for 60 min in a Tissue Tek TEC (Vogel, Gieβgen). Storage was at 4° C.

Sections of 7 μm were taken on a microtome (Leica, Nussloch, Germany), placed on silanized glass slides and dried for 15 h at 40° C. To remove the paraffin, the tissues were transferred into xylol and rehydrated in seven successive steps for 2 min each in 100%, 95%, 90%, 80%, 70%, 50% and 30% ethanol, 5 min in 0,9% NaCl, 5 min in PBS-buffer. After fixation for 20 min in 4% paraformaldehyde/PBS the sections were washed 2×5 min in PBS, digested with with RNase-free proteinase K (20 μg/ml) at 37° C. for 20 min, washed again in PBS for 5 min. Excess paraformaldehyde was blocked by acetylation with 0,625 ml acetic anhydride in 3,125 ml triethanolamine and 250 ml H₂O for 10 min, and the slides were finally washed for 5 min in PBS. To reduce background, the sections were prehybridized for 1-2 h in hybridisation solution in the absence of the probe.

(L) Hybridisation Solution

10× stock solution of salts: 0,2% polyvinylpolypyrrolidone; 0,2% Ficoll 400; 1,38% NaH₂PO₄, pH 6,8; 50 mM EDTA; 3 M NaCl; 0,1 M Tris, pH 8,0.

100 μl of this stock solution were mixed with 500 μl of formamide, 2 μl 10 mg/ml t-RNA, 200 μl of 50% dextran sulfate, 100 μl 1 M 1,4-dithiothreitol, and 100 μl H₂O.

(M) Preparation of Probes

To remove contaminating free labeled nucleotides from the digitonin-labeled RNA probes, 20 μl aliquots of probe were precipitated by addition of 2,4 μl 4M LiCl and 75 μl 100% ethanol and incubation at −70° C. for 15 h. The precipitate was spun down in a centrifuge, the supernatant removed, and the probe washed in 50 μl of 70% ethanol. The sediment was centrifuged, air-dried and taken up in the hybridisation solution to a final concentration of 5 μg/ml. The probe was denatured for 7 min at 70° C. and applied to the tissue sections. In parallel, a known labeled probe was applied to control sections.

(N) Hybridisation

Prehybridisation buffer was cautiously removed from the slides. 100 μl of hybridisation solution containing the digitonin-labeled RNA probe was applied to each section. Slides were incubated in a moist chamber for 15 h at 45° C.

(O) Posthybridisation

Solutions:

SSC 88,2 g Na-citrate×2H₂O, 175,325 g NaCl in 1000 ml H₂O, adjusted to pH 7.0 with HCl; 2×SSC; 2×SSC/50% formaldehyde; 5×SSC and 5×SSC/50% formaldehyde were prepared accordingly:
NTE-buffer 100 ml 5 M NaCl, 10 ml 1 M Tris pH 8,0, 100 ml 0,5 M EDTA, 790 ml H₂O;
B1 DIG 100 ml 1 M Tris pH 7,5, 30 ml 5 M NaCl, 870 ml H₂O;
Blocking solution 50 ml B1 DIG, 0,5 g blocking reagent, 2 mM Levamisol;
Anti-DIG-FAB-fragment 1:500 dilution in 4 ml B1 DIG; NTMT 10 ml 5 M NaCl, 50 ml 1 M Tris-HCl pH 9,5, 25 ml 1 M MgCl₂, 415 ml H₂O, 500 μl Tween 20;
   Staining solution amount corresponding to anti-DIG-FAB-fragment: NTMT, 2 mm Levamisol, BCIP (5-bromo-4-chloro-3-indolylphosphate), NBT (Nitroblue Tetrazolium)

The hybridisation solution was removed from the slides, the sections were washed 2×5 min in 5×SSC at 25° C., 30 min with 5×SSC/50% formamide while the temperature increased from 37° C. to 60° C., and 60 min in 2×SSC/50% formamide. To remove unbound single-stranded RNA-probe, the sections were then incubated for 15 min at 37° C. in NTE-buffer, unbound probe was digested for 30 min at 37° C. with RNAse A (20 μg/ml) in NTE-buffer, followed by a wash for 15 min at 25° C. in NTE-buffer, and 2×SSC for 30 min at 25° C.

The tissue sections were now treated for 15 min with B1 DIG at 25° C. The reaction was terminated by blocking solution for 60 min at 25° C. The blocking solution was removed, anti-DIG-FAB-fragments in blocking solution were added and incubated for 15 h at 4° C.

The sections were washed for 10 min with B1 DIG and NTMT with 2 mM Levamisol at 25° C. Staining solution was added and the slides were incubated in the dark. A red-brown staining results. When the desired intensity is reached, the reaction is stopped by washing with PBS. For conservation, the slides were treated 2×5 min with 70% and 90% ethanol, followed by 2×10 min 100% ethanol and xylol. Finally the stained sections were covered with Entellan. Micrographs were viewed and documented using the foto microscope of the Arcturus microdissector.

(P) Treatment of Cells with Methyltransferase Inhibitors

Treatment of cells with methyltransferase inhibitors was carried out as described by Widschwendter et al., J. Nat. Cancer Inst. 92 (2000), 826-832). All-trans-retinoic acid (ATRA) and 5-aza-deoxycytidine (Aza-CdR) were from Sigma Chemical Co. (St. Louis, Mo.). 1 mM solutions were prepared in dimethyl sulfoxide and diluted in culture medium. Cells were incubated either for 5 days, with 2.5 mM Aza-CdR, for 24 h with 1 mM ATRA, or for 5 days with 2.5 mM Aza-CdR followed by 24 h with 1 mM ATRA, or left untreated. They were detached with trypsin (0.05%)-EDTA (0.02%) in Dulbecco's phosphate-buffered saline (PBS), washed, and pelleted.

(Q) Cloning of the GFP-Drop1 Aminoterminal Domain Fusion Construct for Expression in Mammalian Cells

The 5′ region of the ORF, encoding aar 1-396 (Drop1 ) the two calponin homology domains, was amplified using the primers 5′-GTCGACGATGGCAACCTCCAGAGGGGCCTCCCG-3′ with a Sal I restriction site as upper primer (U278) and 5′-GGGCCCTGGTGCCCAGAGGTGCAGGAAGAGA-3′ with an Apa I restriction site as lower primer (L1492) and a previously sequenced overlap clone (H) on the basis of cDNA from the SKOV-3 cell line as template (annealing at 68 øC, 25 cycles). The PCR product was cloned into pDrive for amplification and sequencing, cut out with Sal I and Apa I, and cloned into pEGFP-C3 (Clontech, GenBank Accession # U57607), behind a GFP-domain. Correct insertion was confirmed by sequencing. 5-10×104 COS-7 cells/well were grown in DMEM, containing 10% fetal calf serum, 1% L-glutamine and 1% nonessential amino acids, for 24 h in Lab-Tek chambered cover glasses, transfected with 0,75-1.5_g of DNA/well mixed with 3 to 6_{—}1 FuGENE 6 (Roche). After 24 h cells were observed under a Zeiss Axiovert S100 TV microscope (excitation at 485 nm, filter 485/20, —emission at 515 nm, filter 530/30, dichroic mirror 505) using a 20× objective and a Plan-Neofluar oil objective with 63× magnification. Fluorescence images were recorded for 100 to 250 msec with 30% gain.

(R) Cloning of the Carboxyterminal Spectrin-like Domain

The carboxyterminal spectrin-like domain of Drop1 was selected as an antigen. cDNA encoding that region was obtained using cDNA from CAOV-3 tumor cells as template, 5′-GATATCATGTATTTAGATGCGGTCCACGAGTTC-3′, containing an EcoR V restriction site as an upper primer, and 5′-GTCGACTGGGGACCCTTGATTTAGAGGTTAACTT-3′, containing a Sal I restriction site as lower primer. This primer binds to a sequence in the 3′ UTR of Drop1, which in the enaptin gene is located in a putative intron. The PCR product (annealing at 64 øC, 45 cycles) was cloned into TopoTA for amplification and sequencing, cut out with EcoR V and Sal I, and cloned into pET32a for expression in E. coli BL21(DE3).

(S) Expression of the Recombinant Protein in E. coli and its Purification from Cell Lysates

Bacteria were induced at 25 øC for 3 h with 1 mM IPTG, harvested by low-speed centrifugation, washed in PBS containing protease inhibitor cocktail (Roche), swollen on ice in hypotonic 50 mM sodium phosphate pH 8.0 containing 1 mg/ml lysozyme, supplemented by EDTA-free protease inhibitor cocktail, and lysed by ultrasonication. DNA was digested with 1 U/ml of benzonase (Merck) for 30 min at 25 øC. Upon addition of 300 mM NaCl the lysate was centrifuged in an ultracentrifuge at 226,000×g for 60 min. The His-tag-containing proteins in the supernatant were bound to Ni-NTA Superflow in a column of 1,6 cm_{—}×10 cm (Qiagen). Unbound material was removed by 50 mM Na phosphate, 300 mM NaCl, 20 mM imidazole, and the bound protein was eluted with the same buffer containing 150 mM imidazole. Fractions of 1 ml were taken and aliquots were checked for protein bands by gel electrophoresis.

Fractions of interest were pooled, diluted ten-fold with 25 mM Tris.HCl pH7.5, loaded onto a MonoQ HR 5/5 column 0.5 cm_{—×}5 cm (Amersham Bioscience) and a gradient was run from the loading buffer with 30 mM NaCl to the same buffer containing 1M NaCl. The relevant fractions were pooled, concentrated, and the buffer was changed to 50 mM Tris pH 8.0, as required for cleavage with enterokinase (Invitrogen). With 10 U of this protease/mg of protein the thioredoxin fused to the C-terminal Drop1-fragment was cleaved for 16 h at 8 øC. The enterokinase was removed by EK-Away resin (Invitrogen). The solution was diluted with 50 mM sodium phosphate containing 500 mM NaCl pH 8.0 as the starting buffer for a Ni-NTA column (1 cm_{—}×5.5 cm), to which the thioredoxin containing the His-tag was bound, while the C-terminal Drop1 domain remained in the flowthrough. It was concentrated in a Savant Speedvac Plus vacuum centrifuge and for the immunization of mice and rabbits the buffer was changed by passage over a NAP25 column (Pharmacia). Purity and quantity of the protein (0.6 mg/ml) were checked by gel electrophoresis on a 15% Kornberg gel.

(T) Generation of Monoclonal Antibody (in Particular DROP1-2.9)

Female BALB/c mice were immunized with the purified C-terminal spectrin-like domain. For the first immunization the protein emulsified in complete Freund_s adjuvant was injected subcutaneously at 4 sites at the flank. Additional five immunizations were given intraperitoneally in intervals of two weeks consisting of the purified protein diluted in sterile PBS. Three days after the last challenge, immune spleen cells were fused to Ag8.653 mouse myeloma cells according to standard procedures. Supernatants of growing hybridomas were screened for binding to the protein by enzyme-linked immunosorbent assay (ELISA). Positive samples were further analyzed by immunocytology using cytocentrifuge preparations of SK-OV-3 ovarian carcinoma cells. Thus, hybridoma DROP1-2 was identified and subsequently cloned by limiting dilution. Clone DROP1-2.9 was selected according to the same assays mentioned above. It secretes an IgG1 kappa antibody as demonstrated by a mouse isotype-specific ELISA.

(U) Staining of Cytospins of SKOV-3 Cells

SKOV-3 cells were spun down on a coverslip and permeabilized and fixed for 10 min at −20 øC in acetone. After drying they were incubated with peroxidase-coupled anti-mouse IgG-Fc for 30 min at 25 øC: A stock solution of 8 mg/ml 3-amino-9-ethylcarbazole (Sigma) in dimethyl formamide was prepared. For use it was diluted 20-fold with 0.1 m Na acetate pH 5.2 and 2_l/ml of H2O2 were added This solution was used for generating a red stain with the peroxidase within 5-10 min. The reaction was stopped by two washes with PBS.

### EXAMPLE 2

### Differential Display PCR

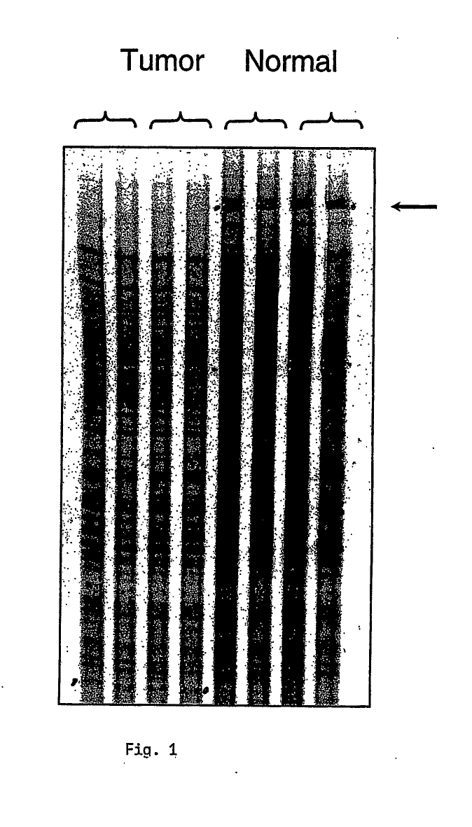

In an analysis of differential gene expression of ovarian carcinomas a 691 bp cDNA fragment was found which was downregulated in an ovarian carcinoma (FIG. 1). The band labeled with an arrow shows a PCR product in normal tissue absent from the tumor. A specifically designed arbitrary primer and a G-anchor primer had been used for this PCR. The product was eluted from the gel, re-amplified, cloned into the TOPO TA vector and sequenced.

### EXAMPLE 3

### Validation of Differential Display Data by Quantitative PCR Using cDNA from Tissues of Patients

Using gene specific primers, normal and ovarian carcinoma tissue of eight individual patients was analyzed by quantitative PCR for differences in expression (Table 1). In all patients Drop1 was expressed at a higher level in the healthy tissue, the ratio of expression between normal tissue and carcinoma varying from 14:1 to 210:1. In addition, it was shown that healthy fallopian tube epithelium is, in this case, a suitable reference tissue for analyzing expression differences in ovarian carcinomas, since microdissected epithelium cells from healthy fallopian tube and ovarian surface epithelium express Drop1 at a comparable level. The ratio of fallopian tube to ovarian surface epithelium was 0.95.

TABLE 1
Drop1 expression level in tissue pairs from individual
patients and in ovarian carcinoma cell lines

| | normal | | | | |
|---|---|---|---|---|---|
| patient | microdissected | | Ratio N:C | OVCA cell lines | |
| 82-84 | tube epithelium | ovarian | 0.95 | NIH: OVCA R-3 | 43 pM |
| | 20 nM | surface | | | |
| | | epithelium | | | |
| | | 21 nM | | | |
| | cDNA from swabs | carcinoma | | MDAH 2774 | 34 pM |
| | normal | | | | |
| 30 | 336 nM | 5.8 nM | 58 | CAOV-3 | 5.0 nM |
| 31 | 1020 nM | 6.9 nM | 148 | SKOV-3 | 11 nM |
| 44 | 94 nM | 0.65 nM | 140 | HEST | 0.26 nM |
| 48 | 320 nM | 23 nM | 14 | ES-2 | 51 pM |
| 50 | 300 nM | 2.3 nM | 130 | GG | 19 pM |
| 53 | 928 nM | 4.4 nM | 210 | MT | 66 pM |
| 93 | 3.4 nM | Neg | 130 | | |
| 100 | 19 nM | Neg | 48 | | |
| BL | ductal epithelium | ductal | 1.0 | MCF-7 | 20 pM |
| | 8.5 nM | invasive | | | |
| | | carcinoma | | | |
| | | 8.5 nM | | | |
| | | | | HeLa | 5.3 nM |

Calculated to the level after 38 PCR cycles; concentration expressed as nM.

### EXAMPLE 4

### Expression of Drop1 in Tumor Cell Lines and in Frozen Sections from Tumors

Ovarian carcinoma cell lines were also analyzed for Drop1 expression (Table 1). Quantitative PCR with pairs of tissues from individual patients was performed with normalized cDNA derived without amplification from healthy tube epithelium and ovarian carcinoma. As expected, expression was low in most cell lines. However, two cell lines, SKOV-3 and CAOV-3 had a normal expression level of approximately 5 nM after 38 PCR cycles. In addition, cDNA was prepared from frozen sections of ovarian and mammary carcinomas from individual patients. In these specimens, Drop1 was downregulated in 6 out of 12 ovarian carcinomas and in eight out of 16 mammary carcinomas (Table 2).

TABLE 2
Drop1 expression levels in frozen sections of tumors from
individual patients

| Patient | | | Patient | Drop1 level | |
|---|---|---|---|---|---|
| Ovarian CA | Drop1 level | | Mammary CA | Drop1 level | |
| 829 | 2.7 | nM | 1170 | 0.020 | nM |
| 895 | 3.1 | nM | 1177 | neg. | |
| 897 | 0.011 | nM. | 1179 | neg. | |
| 1111 | 3.0 | nM | 1181 | 150 | nM |
| 1117 | neg | | 1182 | neg | |
| 1130 | 7.2 | nM | 1184 | neg | |
| 1131 | 0.005 | nM | 1188 | 31 | nM |
| 1160 | 3.8 | nM | 1189 | 24 | nM |
| 1161 | 0.012 | nM | 1191 | 11 | nM |

| | | | | |
|---|---|---|---|---|
| 1172 | 0.019 | nM | 1193 | neg. |
| 1174 | 0.014 | nM. | 1194 | neg. |
| 1178 | 3.8 | nM | 1195 | neg. |

| | | |
|---|---|---|
| 1198 | 3.0 | nM |
| 1202 | 5.3 | nM |
| 1203 | 4.0 | nM |
| 1205 | 4.1 | nM |

Results of quantitative PCR using normalized cDNA without amplification from ovarian carcinoma cell lines, from frozen ovarian carcinomas and from mammary carcinomas; drop level calculated for 38 cycles.

### EXAMPLE 5

### Cancer Profiling Array

Using a cancer profiling array the differential expression of Drop1 was analysed in pairs of tissues in additional 14 patients (FIG. 3). On this array (Array I), cDNA from normal ovarian tissue (N) and carcinoma tissue (C) has been spotted in an amount normalized to equal ubiquitin cDNA concentration. The tumors were 12 adenocarcinomas, one mucinous borderline tumor and one clear cell carcinoma. Upon hybridisation with the radioactive Northern probe, in 12 patients the expression of Drop1 was distinctly lower in the tumor. For two patients, cDNA prepared from metastatic tissue was also spotted and featured a similar level of expression as the corresponding primary tumor.

Tissue pairs from patients with other types of carcinomas were likewise analyzed for Drop1 expression. It was significantly lower than in the corresponding normal tissue in 85% of the mammary carcinomas, stomach, lung, kidney and thyroidea carcinomas. Colon and rectum carcinomas have a low expression level with a less distinct pattern, and for cervix, pancreas, and small intestine the number of cases is not representative.

An autoradiogram of another cancer profiling array (Array II; BD Biosciences Clontech, Cat#S2555; Lot # 2060252) hybridized with 3R9 as in FIG. 3 is shown in FIG. 3a. This array includes normalized cDNA from 154 tumor and corresponding normal tissues from individual patients, amplified using BD SMART technology. cDNA from nine cancer cell lines is included at the lower right. In addition to supporting the data obtained with cancer profiling array I these results extend the observation of downregulated Drop1 to cervix, vulva, and pancreas carcinomas.

### EXAMPLE 6

### Regulation of Drop1 by Methylation of the Promoter

The possibility that promoter methylation might downregulate Drop1 was investigated. Treatment of cell lines exhibiting a low level of Drop1 with the demethylating agents AZA (5-aza-cytidine) and ATRA (all-trans-retinoic acid) resulted in an increase of Drop1 mRNA by an order of magnitude (Table 3). No increase of expression was observed in SKOV-3, a cell line with a physiological level of Drop1. This indicates that the lower level of Drop1 expression in cell lines GG and M130 was caused by methylation of the Drop1 promoter.

TABLE 3
Expression of downregulated Drop1 in cells treated with
inhibitors of gene methylation

| Cells | in medium | in Aza | in ATRA | In Aza + ATRA |
|---|---|---|---|---|
| GG | 5.2 × 10⁻⁵ | 4.7 × 10⁻⁵ | 1.3 × 10⁻⁴ | 7.7 × 10⁻⁴ 15 fold |
| | | | 2.5 fold | |
| M130 | 1.0 × 10⁻³ | 1.0 × 10⁻³ | 1.8 × 10⁻² 18 fold | 5.6 × 10⁻⁴ |
| SKOV-3 | 2.2 × 10⁻⁴ | 2.4 × 10⁻⁴ | 2.2 × 10⁻⁴ | 1.9 × 10⁻⁴ |

Aza: 5′-aza-2′-deoxycytidine, ATRA: all-trans-retinoic acid
$Ratios are \frac{[ 31 N \text{/} GDOC \text{/} 1 ]}{[ G 3 PDH ]} , measured in the LightCycler$

### EXAMPLE 7

### In Situ Hybridisation

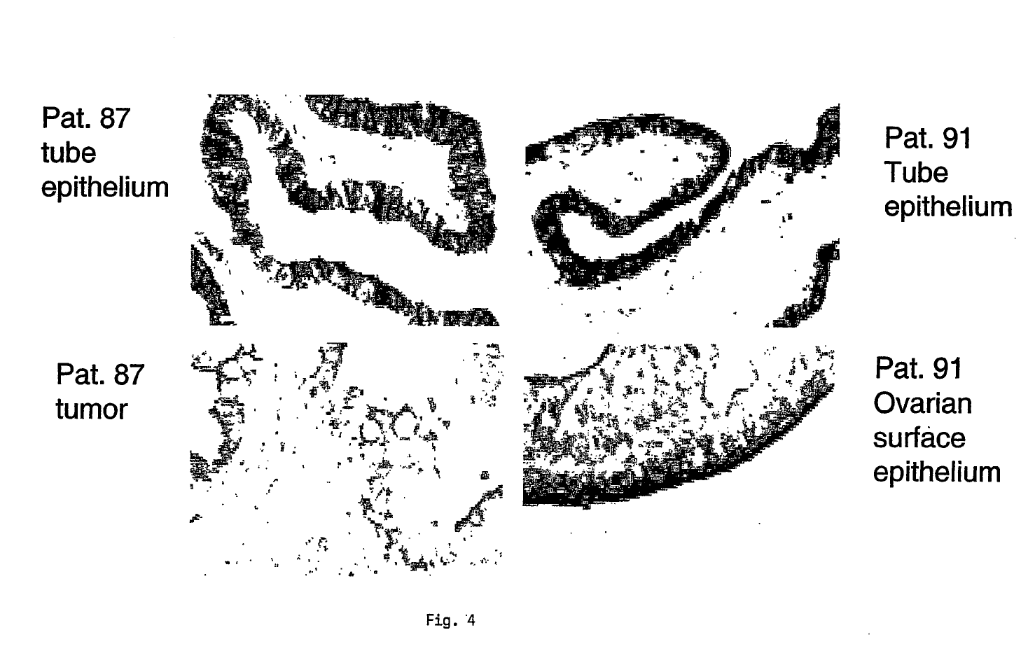

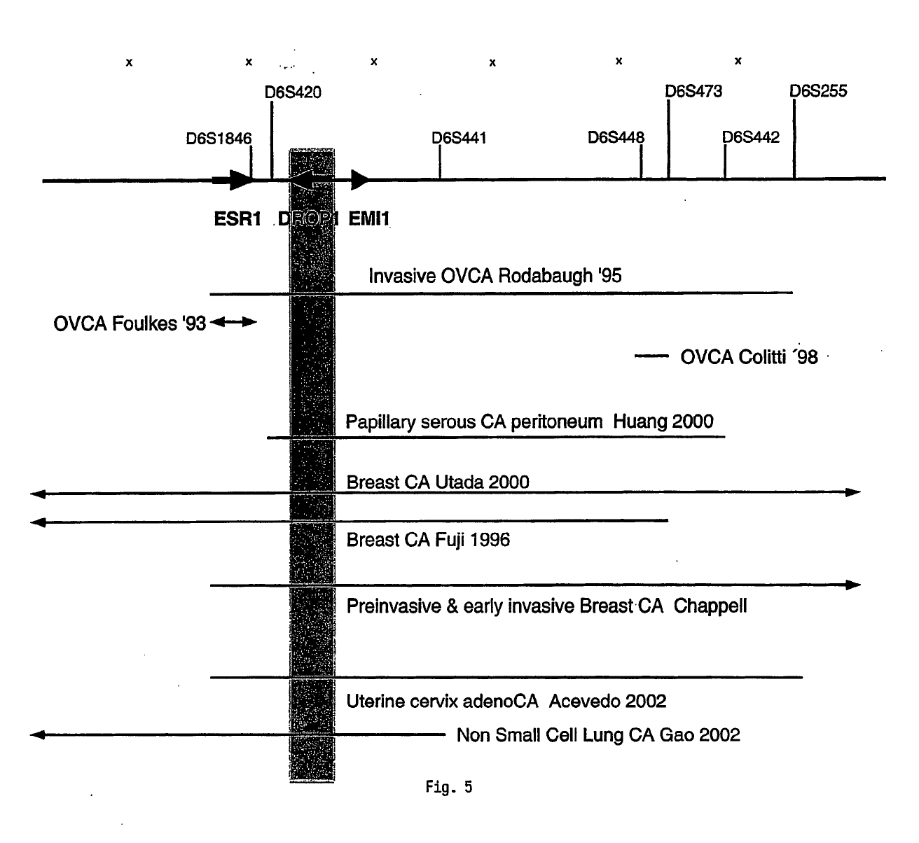

Localization of the mRNA of Drop1 in healthy ovarian tissue, situ hybridisation (FIG. 4). Drop1 was specifically expressed in epithelium of the healthy ovary and at a comparable level in the epithelium of a healthy fallopian tube. Other cell types of these organs do not express Drop1. This figure again shows that tube epithelium in this case is a suitable reference tissue and that the difference in expression of Drop1 is not based on the characteristics of the tube epithelium.

### EXAMPLE 8

### Multi-Tissue-Northern Blot

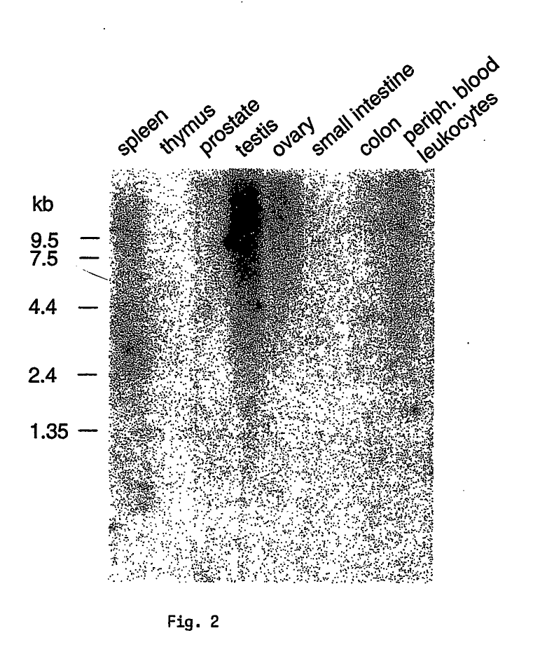

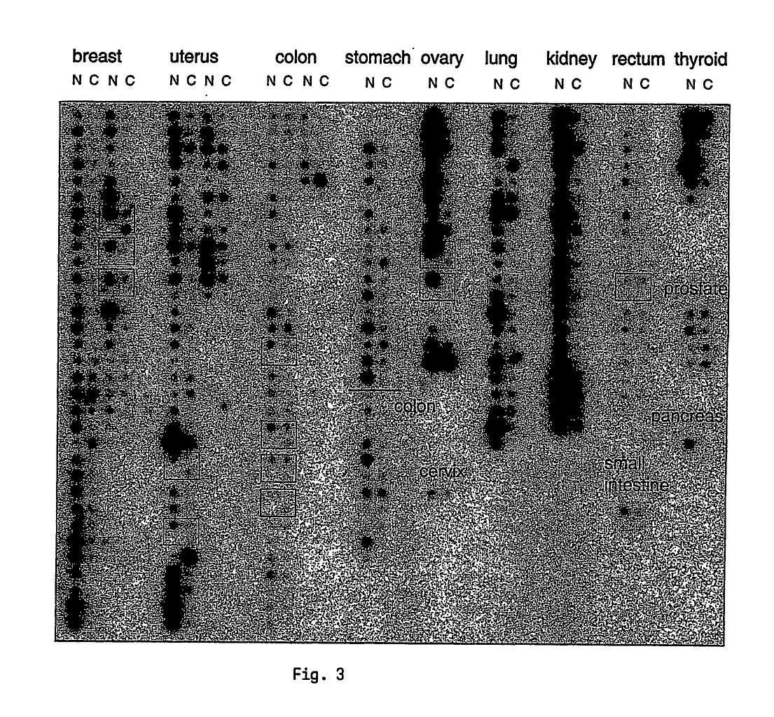

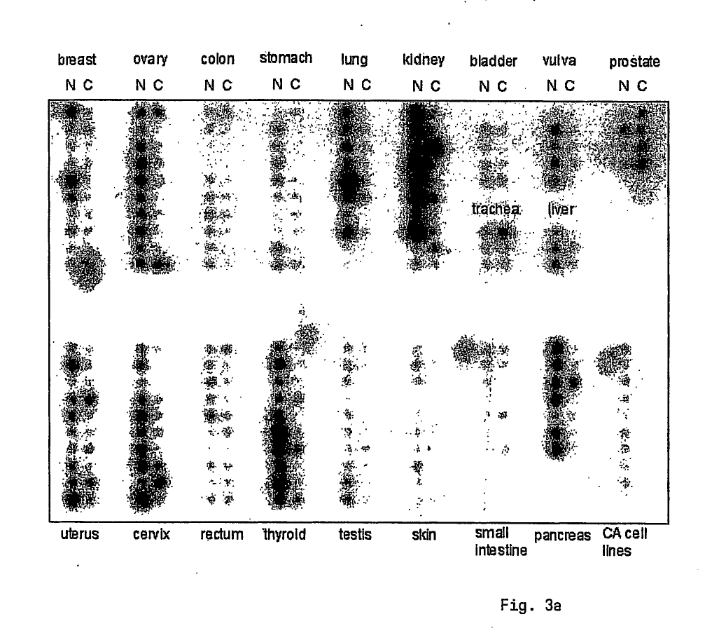

It could be demonstrated (FIG. 2), that Drop1 mRNA is present in testes (FIG. 2; slot 4) and in ovary (FIG. 2; slot 5) in a multi tissue Northern Blot. The remaining tissues yielded only a weak signal or nor signal at all. The smear may be caused by differentially used exons resulting in mRNAs of different lengths.

### EXAMPLE 9

### Completion and Characteristics of the Drop1 Sequence

The sequence of the differential display fragment had featured an open reading frame. By “RACE-walking” using a commercial ovarian RACE library and by validating the resulting RACE clones using overlap PCR on the basis of one ovarian carcinoma cell line that did express the Drop1 fragment, an open reading frame of 9,099 bp (FIG. 6B; sequence details) could be completed. The encoding gene comprises 57 exons in chromosome region 6q25. Upon completion of this sequence a cDNA with a much larger ORF for enaptin was submitted to the data bases (Accession no. AF535142). Exon usage for Drop1, however, was different from that of enaptin in that (a) exon 5 is 36 bp longer than predicted, (b) exon 7 (21 bp) is not used and (c) exon 12 is used in a commercial ovarian cDNA library, yet not in the ovarian carcinoma cell line CAOV-3. It contains the start codon for a putative human homologue of rat cpg2. Exon 58 is longer in Drop1 than predicted, In all clones derived from OVCA-R3 it contains a stop codon immediately following the predicted transition to exon 59. 2,5 kb of the genomic sequence following this stop codon are found in the Drop1 mRNA as 3′ UTR. In contrast, an ovarian RACE library yielded clones for both variants: an extended exon 58 with the stop codon for Drop1 as well as the transition to exon 59.

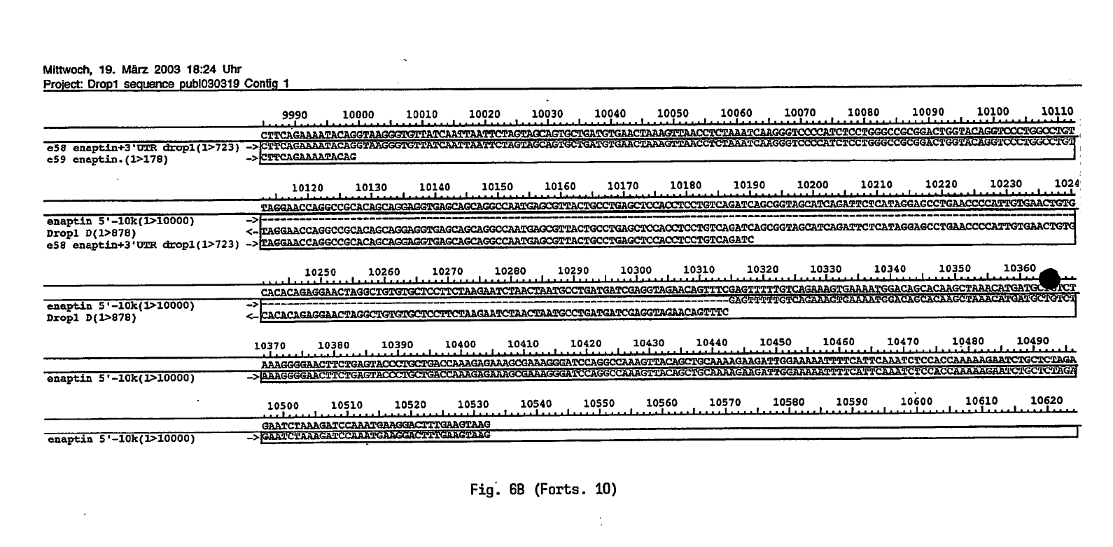

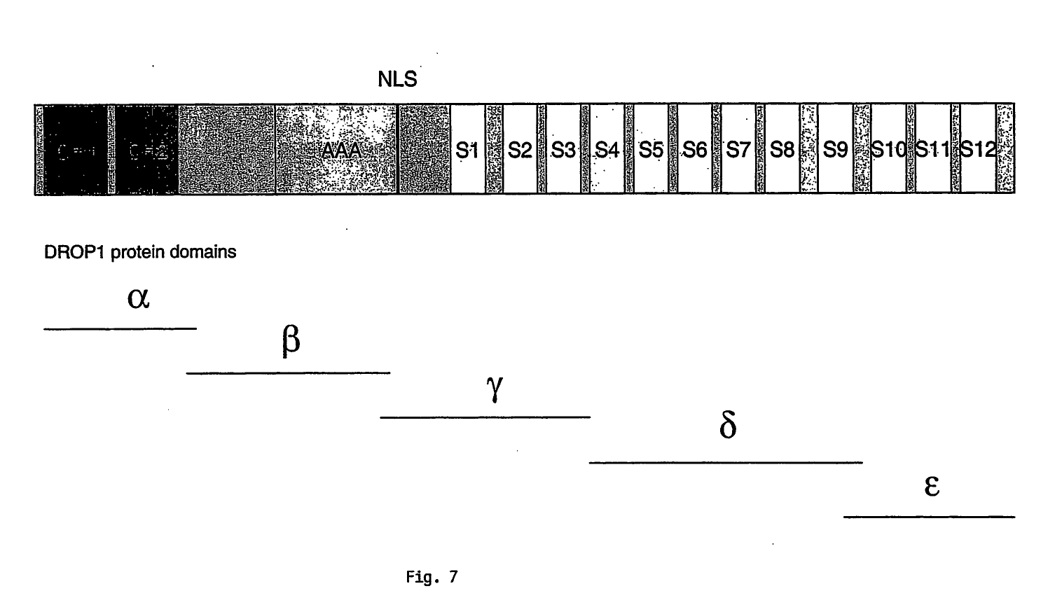

Drop1 encodes a protein of 349 kDa (FIG. 10) that is structurally similar to dystrophin. At the N-terminus it features 2 calponin homology domains followed by an ATPase-domain and 12 spectrin-like domains (FIG. 7). The homologues of these structures are known to bind other intracellular or transmembrane proteins.

### EXAMPLE 10

### Cloning and Expression of a Drop1-GFP Fusion Construct

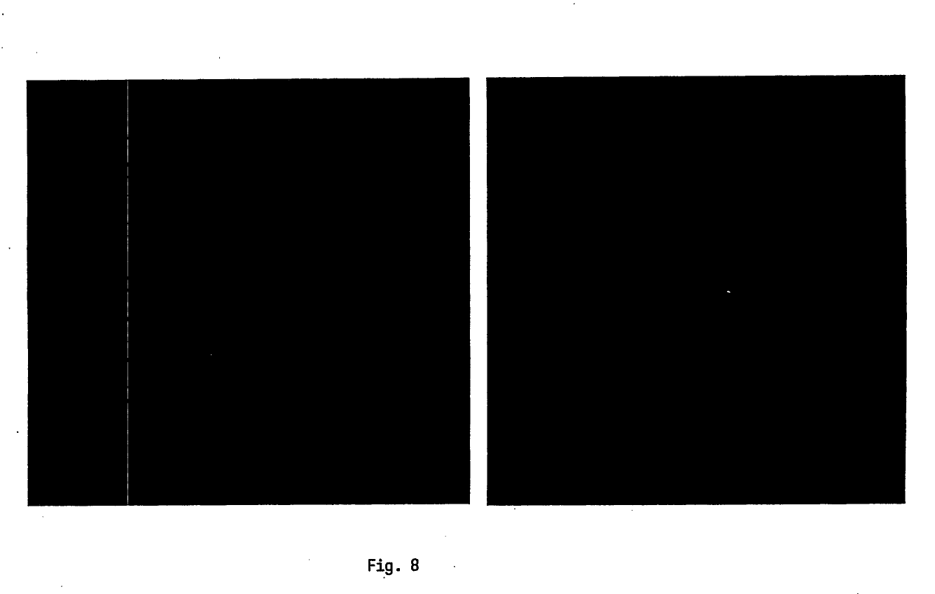

The 5′ fragment, Drop1α, containing the two calponin homology domains of the open reading frame, was cloned into the eukaryotic expression vector pEGFP-C3. Transfected cells show a distinct fluorescence of nuclei (FIG. 8), suggesting a nuclear association of the N-terminal region.

### EXAMPLE 11

### Raising of a Monoclonal Antibody and Detection of the Antigen in Cells and on Immunoblots

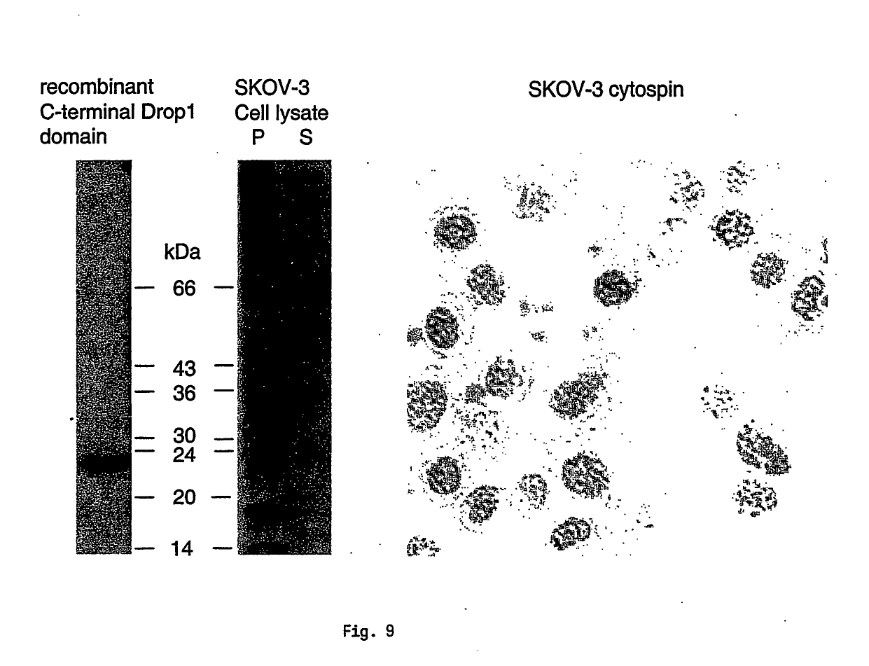

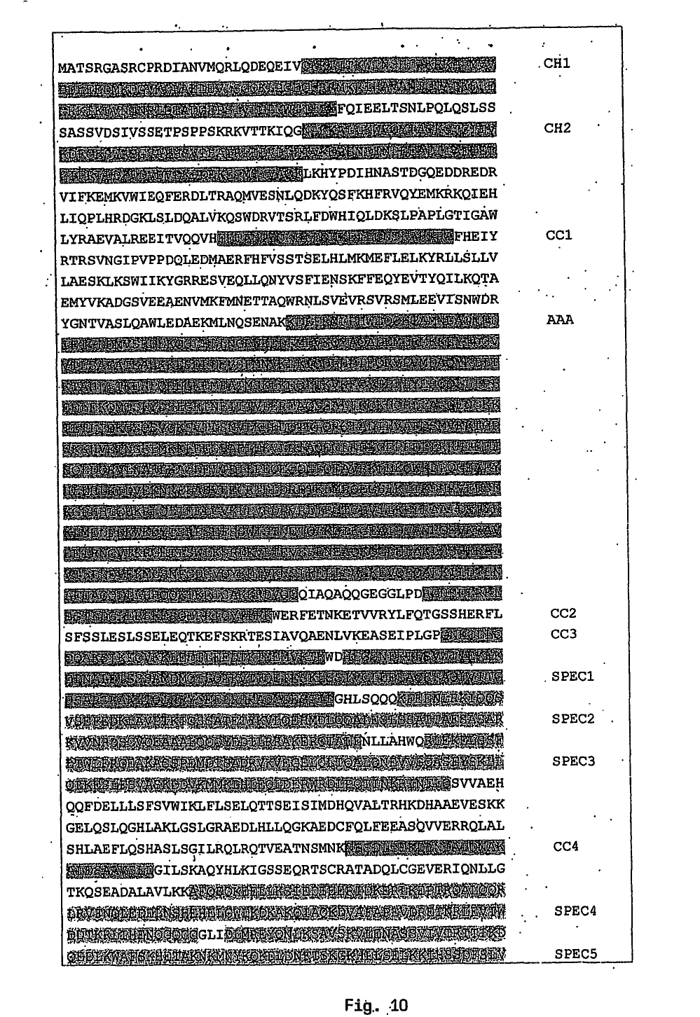

A C-terminal recombinant protein fragment, amino acid residues 2827-3032, was produced in bacteria, isolated and purified to serve as an antigen for generation of a monoclonal antibody. A clone that recognized the recombinant protein on an immunoblot also identified a high molecular weight band in cell lysates of SKOV-3 and stained nuclei of the same cells (FIG. 9). Additional smaller protein bands in the pellet of the cells presumably are Drop1 degradation products.

### EXAMPLE 12

### Immunohistochemistry on Frozen Human Tissue Sections

2 cm-thick cryosections were immediately fixed in ice-cold acetone for 10 min, air-dried and incubated for 1 h with monoclonal antibody against Drop1. For detection of bound antibody, a goat-anti-mouse Ig conjugated to peroxidase labeled-dextran polymer in Tris-HCL containing carrier protein (EnVision, Dako, Denmark) was used. 3-amino-9-ethyl-carbazole (Sigma, St. Louis, Mo.) was used as the substrate. Hematoxylin was used as a counterstain. Controls were performed by omitting the monoclonal antibody and yielded negative results.

The murine monoclonal antibody directed against the C-terminal domain of Drop1 was used for immunohistochemistry on frozen tissue sections of normal premenopausal human ovary (FIGS. 11 and 12) and samples of human ovarian carcinoma (FIGS. 13 and 14). There was a cytoplasmic reactivity of ovarian surface epithelium and of a few scattered ovarian stromal cells of fibroblastoid morphology. Staining intensity of stained cells was always strong in normal ovary, All other types of ovarian epithelia (follicle epithelia, lutein and theca cells etc.) were negative. With the exception of the above-mentioned Drop1-expressing stroma cells all other sorts of mesenchymal cells, leucocytes and erythrocytes included, were negative. In the ovarian carcinoma sample, the carcinoma cells stained weakly for Drop1 while a subset of stromal cells with fibroblastoid morphology stained strongly, like in normal ovary.

1. An isolated nucleic acid encoding a human dystrophin-related polypeptide Drop1 or an isolated polypeptide exhibiting a biological property of Drop1, wherein the nucleic acid:
(a) encodes the amino acid sequence in SEQ ID NO:30;
(b) consists of the nucleotide sequence of any of SEQ ID NOS:9-29;
(c) consists of a nucleotide sequence which differs from the sequence of (b) due to the degeneracy of the genetic code; or
(d) consists of a nucleotide sequence complementary to the sequence specified in (b) or (c).

2. A recombinant vector comprising the nucleic acid molecule of claim 1.

3. The recombinant vector of claim 2 wherein the nucleic acid molecule is operatively linked to one or more regulatory elements allowing transcription and synthesis of a translatable RNA in a prokaryotic or eukaryotic host cell.

4. A recombinant host cell which contains the recombinant vector of claim 2.

5. The recombinant host cell of claim 4 or 35, which is a mammalian cell, a bacterial cell, an insect cell or a yeast cell.

6. A non-human transgenic animal characterized by loss of Drop-1 function.

7. The transgenic non-human animal of claim 6 comprising at least one inactivated wild type allele of the corresponding Drop1 encoding gene.

8. An isolated polypeptide encoded by a nucleic acid molecule of claim 1.

9. A method of making a polypeptide, comprising:
(a) culturing the recombinant host cell of claim 4 under conditions such that the polypeptide is expressed; and
(b) recovering the polypeptide.

10. A polypeptide made by the method of claim 9.

11. An antibody that binds to the polypeptide of claim 8.

12. The nucleic acid of claim 1, which is detectably labeled.

13. The nucleic acid of claim 12, wherein the label comprises a radioisotope, a bioluminescent compound, a chemiluminescent compound, a fluorescent compound, a metal chelate, or an enzyme.

14. A method for identifying an activator of a human dystrophin-related polypeptide Drop1 comprising:
(a) incubating a candidate compound with a polypeptide of claim 8;
(b) assaying for a biological activity of the polypeptide; and
(c) determining if a biological activity of the polypeptide has been altered relative to that of the polypeptide when not being incubated with the candidate compound.

15. A pharmaceutical composition comprising:
(a) a nucleic acid encoding a Drop1 polypeptide that comprises the amino acid sequence as depicted in SEQ ID NO:30;
(b) a nucleic acid comprising the nucleotide sequence as depicted in of any of SEQ ID NOS:9-29;
(c) a nucleic acid encoding a polypeptide the amino acid sequence of which shows at least 65% identity to the amino acid sequence of the polypeptide encoded by a nucleic acid specified in (a) or (b);
(d) a nucleic acid, the sequence of which differs from the sequence of a nucleic acid molecule of (a) to (c) due to the degeneracy of the genetic code;
(e) a nucleic acid, which represents a fragment of a nucleic acid specified in (a) to (d); or
(f) a nucleic acid complementary to the nucleic acid molecule specified in (a) to (e),
(g) an isolated polypeptide encoded by a nucleic acid of (a) to (f) above;
(h) a recombinant vector comprising the nucleic acid molecule of
(a) to (f) above; or
(i) an antibody that binds to the polypeptide of (g) above.

16. (canceled)

17. A diagnostic kit or array useful for the detection or characterization of a tumor or a predisposition to such a tumor, comprising a nucleic acid molecule as defined in claim 15, a polypeptide as defined in claim 15 or an antibody as defined in claim 15.

18. (canceled)

19. (canceled)

20. (canceled)

21. A method of making a polypeptide, comprising:
(a) culturing the recombinant host cell of claim 35 under conditions such that the polypeptide is expressed; and
(b) recovering the polypeptide.

22. A polypeptide made by the method of claim 21.

23. An antibody that binds to the polypeptide of claim 22.

24. An antibody that binds to the polypeptide of claim 10.

25. A method for identifying an activator of a human dystrophin-related polypeptide Drop1 comprising:
(a) incubating a candidate compound with a polypeptide of claim 22;
(b) assaying for a biological activity of the polypeptide; and
(c) determining if a biological activity of the polypeptide has been altered relative to that of the polypeptide when not being incubated with the candidate compound.

26. A method for identifying an activator of a human dystrophin-related polypeptide Drop1 comprising:
(a) incubating a candidate compound with a polypeptide of claim 10;
(b) assaying for a biological activity of the polypeptide; and
(c) determining if a biological activity of the polypeptide has been altered relative to that of the polypeptide when not being incubated with the candidate compound.

27. A method for treating a cancer comprising administering to a subject in need thereof an effective amount of a nucleic acid molecule as defined in claim 15.

28. A method for treating a cancer comprising administering to a subject in need thereof an effective amount of a polypeptide as defined in claim 15.

29. A method for treating a cancer comprising administering to a subject in need thereof an effective amount of a recombinant vector as defined in claim 15.

30. A method for treating a cancer comprising administering to a subject in need thereof an effective amount of an antibody as defined in claim 15.

31. A method for treating a cancer comprising administering to a subject in need thereof an effective amount of an activator/agonist obtainable by the method of claim 14, 25 or 26.

32. A method for diagnosing and/or characterizing a tumor or a predisposition to such tumor comprising determining whether Drop1 gene expression is downregulated in the tumor compared to benign tissue, wherein downregulation of the Drop1 gene indicates malignant disease in the tumor or predisposition to a malignant tumor.

33. The method of claim 32, wherein the tumor is an ovarian, mammary, stomach, kidney, thyroid, cervical, pancreas, testis or lung carcinoma.

34. A method for treating a cancer comprising administering to a subject in need thereof an effective amount of the vector of claim 2.

35. A recombinant host cell which contains the recombinant vector of claim 3.

36. The polypeptide of claim 8, 10 or 22, which is detectably labeled.

37. The polypeptide of claim 36, wherein the label comprises a radioisotope, a bioluminescent compound, a chemiluminescent compound, a fluorescent compound, a metal chelate, or an enzyme.

38. The antibody of claim 11, 23 or 24, which is detectably labeled.

39. The antibody of claim 38, wherein the label comprises a radioisotope, a bioluminescent compound, a chemiluminescent compound, a fluorescent compound, a metal chelate, or an enzyme.
